SEQUENCE LISTING (46 sequences)

SEQ ID NO: 1 - DNA, 20 bp, Artificial Sequence
  Synthetic gRNA 1
agaatttgttcactgagcca

SEQ ID NO: 2 - DNA, 20 bp, Artificial Sequence
  Synthetic gRNA 2
aattctgcaatcctcactct

SEQ ID NO: 3 - DNA, 20 bp, Artificial Sequence
  Synthetic gRNA 3
ccctgccagtgtaacctcca

SEQ ID NO: 4 - DNA, 20 bp, Artificial Sequence
  Synthetic gRNA 4
ctgagccatggaggttacac

SEQ ID NO: 5 - DNA, 127 bp, Artificial Sequence
  Synthetic ssODN 1
ggcttaggtgtgatcattgcaattttggatttaaatttctccgaagctttaatgatgttggatgtgagccctgccagtgtaacctccatggctcagtgaacaaattctgcaatcctcactctgggca

SEQ ID NO: 6 - DNA, 127 bp, Artificial Sequence
  Synthetic ssODN 2
tacaattggtgacatctaacccataaaagttttctctgcaggtgtcacactgaagtcctttggcttcttttttgcactcacactggccagagtgaggattgcagaatttgttcactgagccatggag

SEQ ID NO: 7 - DNA, 20 bp, Artificial Sequence
  Synthetic gRNA 1S
agaatttgttcactgagccg

SEQ ID NO: 8 - DNA, 99 bp, Artificial Sequence
  Synthetic sequence 5′-3′ from Exon 13 of USH2A - FIG 1A
cgaagctttaatgatgttggatgtgagccctgccagtgtaacctccatggctcagtgaacaaattctgcaatcctcactctgggcagtgtgagtgcaaa

SEQ ID NO: 9 - DNA, 99 bp, Artificial Sequence
  Synthetic sequence 3′-5′ from Exon 13 of USH2A - FIG 1A
gcttcgaaattactacaacctacactcgggacggtcacattggaggtaccgagtcacttgtttaagacgttaggagtgagacccgtcacactcacgttt

SEQ ID NO: 10 - DNA, 127 bp, Artificial Sequence
  Synthetic FIG 1D ssODN 1
ggcttaggtgtgatcattgcaattttggatttaaatttctccgaagctttaatgatgttggatgtgagccctgccagtgtaacctacatggctcagtgaacaaattctgcaatcctcactctgggca

SEQ ID NO: 11 - DNA, 31 bp, Artificial Sequence
  Synthetic sequence complementary to a part of SEQ ID NO 10 in FIG 1D
ccatggctcagtgaacaaattcttcaatcct

SEQ ID NO: 12 - DNA, 36 bp, Artificial Sequence
  Synthetic sequence complementary to a part of SEQ ID NO 13 in FIG 1D
gtttaagacgttaggagtgagacccgtcacatcacg

SEQ ID NO: 13 - DNA, 127 bp, Artificial Sequence
  Synthetic FIG 1D ssODN 2
gaggtaccgagtcacttgtttaagacgttaggagtgagaccggtcacactcacgttttttcttcggtttcctgaagtcacactgtggacgtctcttttgaaaatacccaatctacagtggttaacat

SEQ ID NO: 14 - DNA, 10 bp, Artificial Sequence
  Synthetic FIG 2B - B1F11 sequence
  variation 7: /replace=“n in position 7 represents any base selected from A, T, C and G”
  variation 8: /replace=“n in position 8 represents any base selected from A, T, C and G”
  variation 9: /replace=“n in position 9 represents any base selected from A, T, C and G”
  variation 10: /replace=“n in position 10 represents any base selected from A, T, C and G”
atcctcnnnn

SEQ ID NO: 15 - DNA, 19 bp, Artificial Sequence
  Synthetic FIG 2B - B3B8, B3B1 and B2H4 sequences
ctctggccagtgtgagtgc

SEQ ID NO: 16 - DNA, 44 bp, Artificial Sequence
  Synthetic FIG 2C - Heterozygous correction A1
tgaacaaattctgcaatcctcactctggccagtgtgagtgcaaa

SEQ ID NO: 17 - DNA, 39 bp, Artificial Sequence
  Synthetic FIG 2C - Heterozygous correction A2
tgaacaaattctgcaatcctctgggcagtgtagtgcaaa

SEQ ID NO: 18 - DNA, 44 bp, Artificial Sequence; identical to SEQ ID NO: 16
  Synthetic FIG 2C - Homozygous correction A1

SEQ ID NO: 19 - DNA, 44 bp, Artificial Sequence; identical to SEQ ID NO: 16
  Synthetic FIG 2C - Homozygous correction A2

SEQ ID NO: 20 - DNA, 57 bp, Artificial Sequence
  Synthetic FIG 3B - Partial edition of A2 - A1
acctccacggctcagtgaacaaattcttcaatcctcactctgggcagtgtgagtgca

SEQ ID NO: 21 - DNA, 56 bp, Artificial Sequence
  Synthetic FIG 3B - Partial edition of A2 - A2
acctacatggctcagtgaacaaattctgcaatcctcactctgggcagtgtagtgca

SEQ ID NO: 22 - DNA, 57 bp, Artificial Sequence; identical to SEQ ID NO: 20
  Synthetic FIG 3B - INDELs in A2 - A1

SEQ ID NO: 23 - DNA, 55 bp, Artificial Sequence
  Synthetic FIG 3B - INDELs in A2 - first A2
acctccatgctcagtgaacaaattctgcaatcctcactctgggcagtgtagtgca

SEQ ID NO: 24 - DNA, 55 bp, Artificial Sequence
  Synthetic FIG 3B - INDELs in A2 - second A2
acctccatggtcagtgaacaaattctgcaatcctcactctgggcagtgtagtgca

SEQ ID NO: 25 - DNA, 52 bp, Artificial Sequence
  Synthetic FIG 3B - INDELs in A2 - third A2
acctccatgagtgaacaaattctgcaatcctcactctgggcagtgtagtgca

SEQ ID NO: 26 - DNA, 42 bp, Artificial Sequence
  Synthetic FIG 3B - INDELs in A2 - fourth A2
acctccaaattctgcaatcctcactctgggcagtgtagtgca

SEQ ID NO: 27 - DNA, 40 bp, Artificial Sequence
  Synthetic FIG 3B - INDELs in A2 - fifth A2
acctccattctgcaatcctcactctgggcagtgtagtgca

SEQ ID NO: 28 - DNA, 57 bp, Artificial Sequence; identical to SEQ ID NO: 20
  Synthetic FIG 3B - A1 used to repair A2 - A1

SEQ ID NO: 29 - DNA, 57 bp, Artificial Sequence; identical to SEQ ID NO: 20
  Synthetic FIG 3B - A1 used to repair A2 - A2

SEQ ID NO: 30 - DNA, 60 bp, Artificial Sequence
  Synthetic FIG 3C - USH2A-RP-iPSC A1
acctccacggctcagtgaacaaattcttcaatcctcactctgggcagtgtgagtgcaaaa

SEQ ID NO: 31 - DNA, 59 bp, Artificial Sequence
  Synthetic FIG 3C - USH2A-RP-iPSC A2
acctccatggctcagtgaacaaattctgcaatcctcactctgggcagtgtagtgcaaaa

SEQ ID NO: 32 - DNA, 60 bp, Artificial Sequence; identical to SEQ ID NO: 30
  Synthetic FIG 3D - A1

SEQ ID NO: 33 - DNA, 59 bp, Artificial Sequence; identical to SEQ ID NO: 31
  Synthetic FIG 3D - A2

SEQ ID NO: 34 - DNA, 60 bp, Artificial Sequence
  Synthetic FIG 3F - Partial repaired A1 and A2 - A1
acctacatggctcagtgaacaaattcttcaatcctcactctgggcagtgtgagtgcaaaa

SEQ ID NO: 35 - DNA, 59 bp, Artificial Sequence
  Synthetic FIG 3F - Partial repaired A1 and A2 - A2
acctacatggctcagtgaacaaattctgcaatcctcactctgggcagtgtagtgcaaaa

SEQ ID NO: 36 - DNA, 60 bp, Artificial Sequence; identical to SEQ ID NO: 34
  Synthetic FIG 3F - Partial repaired A1 - A1

SEQ ID NO: 37 - DNA, 59 bp, Artificial Sequence; identical to SEQ ID NO: 31
  Synthetic FIG 3F - Partial repaired A1 - A2

SEQ ID NO: 38 - DNA, 59 bp, Artificial Sequence; identical to SEQ ID NO: 31
  Synthetic FIG 3F - A2 used to repair A1 - A1

SEQ ID NO: 39 - DNA, 59 bp, Artificial Sequence; identical to SEQ ID NO: 31
  Synthetic FIG 3F - A2 used to repair A1 - A2

SEQ ID NO: 40 - DNA, 52 bp, Artificial Sequence
  Synthetic FIG 3F - INDELs in A1 - A1
acctcagtgaacaaattcttcaatcctcactctgggcagtgtgagtgcaaaa

SEQ ID NO: 41 - DNA, 59 bp, Artificial Sequence; identical to SEQ ID NO: 31
  Synthetic FIG 3F - INDELs in A1 - A2

SEQ ID NO: 42 - DNA, 60 bp, Artificial Sequence; identical to SEQ ID NO: 34
  Synthetic FIG 3F - INDELs in A2 - A1

SEQ ID NO: 43 - DNA, 51 bp, Artificial Sequence
  Synthetic FIG 3F - INDELs in A2 - A2
acctcagtgaacaaattctgcaatcctcactctgggcagtgtagtgcaaaa

SEQ ID NO: 44 - DNA, 60 bp, Artificial Sequence
  Synthetic FIG 3F - Positive clone MS3F7 A1
acctacatggctcagtgaacaaattctgcaatcctcactctgggcagtgtgagtgcaaaa

SEQ ID NO: 45 - DNA, 59 bp, Artificial Sequence; identical to SEQ ID NO: 35
  Synthetic FIG 3F - Positive clone MS3F7 A2

SEQ ID NO: 46 - DNA, 51 bp, Artificial Sequence
  Synthetic FIG 3G
  variation 47: /replace=“n in position 47 represents any base selected from A, T, C and G”
  variation 48: /replace=“n in position 48 represents any base selected from A, T, C and G”
  variation 49: /replace=“n in position 49 represents any base selected from A, T, C and G”
  variation 50: /replace=“n in position 50 represents any base selected from A, T, C and G”
  variation 51: /replace=“n in position 51 represents any base selected from A, T, C and G”
acatggctcagtgaacaaattctgcaatcctcactctgggcagtgtnnnnn

(19) United States
(12) Patent Application Publication
(10) Pub. No.: US 2022/0213488 A1
(43) Pub. Date: Jul. 7, 2022

(54) CORRECTION OF THE TWO MOST PREVALENT USH2A MUTATIONS BY GENOME EDITING

(71) Applicants: INSERM (istitut National de la SantéRrcherche Médicale), Paris (FR); Université de Montpellier, Montpellier (FR)

(72) Inventors: Carla SANJURJO-SORIANO, Montpellier Cedex 05 (FR); Vasiliki KALATZIS, Montpellier Cedex 05 (FR)

(21) Appl. No.: 17/606,260

(22) PCT Filed: Apr. 29, 2020
(86) PCT No.: PCT/EP2020/061960

§ 371 Date: Oct. 25, 2021

(30) Foreign Application Priority Data
Apr. 30, 2019 (EP) 19305558.9

(51) Int. Cl. C12N 15/113

(52) U.S. Cl. CPC C12N 15/1138; C12N 2320/30; C12N 2310/20

(57) ABSTRACT

The present invention relates to the field of therapeutic treatment of inherited retinal dystrophies, and in particular of syndromic Usher syndrome type 2 and non-syndromic retinitis pigmentosa, by genome engineering. Currently, no treatment is available for this disease, which is caused by mutations in the USH2A gene. By using an in vitro or ex vivo method according to the invention comprising a site-directed genetic engineering system including specific gRNA sequences, the CRISPR technology and a donor nucleic acid sequence as a repair template, the inventors were able to successfully generate USH2A gene corrected iPSCs. The present invention also relates to a system for correcting the two most prevalent USH2A gene mutations in the genome of a cell, such as of photoreceptor cell, of an individual in need thereof comprising specific gRNA sequences, the CRISPR technology and a donor nucleic acid sequence as a repair template.

### FIELD OF THE INVENTION

The present invention relates to the field of therapeutic treatment by genome editing.

In particular, the invention relates to an in vitro or ex vivo method for correcting the two most prevalent USH2A gene mutations in the genome of a patient's cell and to its use as a therapy for inherited retinal dystrophies. Specifically, to treat isolated autosomal recessive retinitis pigmentosa or retinitis pigmentosa in association with hearing loss as part of Usher syndrome type 2.

The invention further relates to a system for correcting USH2A gene mutations in vivo in the genome of an ocular cell and to its use in the treatment of inherited retinal dystrophies, in particular isolated autosomal recessive retinitis pigmentosa or retinitis pigmentosa in association with hearing loss as part of Usher syndrome type 2

### BACKGROUND OF THE INVENTION

Inherited retinal dystrophies (IRD) are a group of neurodegenerative disorders that lead to irreversible blindness and affect approximately 1 in 2000 individuals worldwide (6 and 35). IRDs can be divided into non-syndromic forms, with an isolated retinal phenotype or syndromic forms, in which another organ, in addition to the eye, is affected.

The most common form of syndromic IRD is Usher syndrome (USH), which involves hearing loss, retinitis pigmentosa (RP) and, in some cases, vestibular dysfunction. USH is clinically and genetically heterogeneous and it is the most common cause of inherited deaf-blindness, with a prevalence of approximately 1 in 6000 individuals (16). According to the severity and progression of the disease, three clinical forms of the disease can be distinguished: USH type 1 (USH1), USH type 2 (USH2) and USH type 3 (USH3).

USH2 is the most common form and it is characterized by congenital moderate-severe hearing loss and post-pubertal onset of RP (23). Up to 85% of USH2 patients have mutations in the USH2A gene (38). In addition, 23% of autosomal recessive RP (arRP) cases, with a prevalence of 1 in 4000 individuals worldwide, are also due to mutations in USH2A (22), making USH2A the principal gene responsible for both isolated and syndromic RP (26 and 5).

Over 490 mutations have been identified in USH2A which are distributed throughout the gene (12). The majority of these mutations are sporadic. However there exist two recurrent mutations, c.2276G>T (or p.Cys759Phe) and c.2299delG (or p.Glu767Serfs*21), which are located 22 bp from each other in exon 13. These mutations are the two most prevalent USH2A mutations and together they account for approximately half of the cases of USH2 and arRP. Interestingly, the c.2276G>T mutation has been described as a hypomorphic allele. When c.2276G>T is present in the homozygous or heterozygous state it leads to isolated arRP. In contrast, if the c.2299delG mutation is present in the homozygous state, or as a compound heterozygote with a mutation other than c.2276G>T, it leads to USH2.

Currently, there is no treatment available for patients with mutations in USH2A.

Gene augmentation therapies using adeno-associated viral (AAV) vectors are a promising treatment for monogenic IRDs caused by haploinsufficiency or loss-of-function mutations (10, 13, 19 and 30). However, the major limitation of AAV vectors is their cloning capacity (<4.7 kb), which hinders the transfer of large genes. This limitation has been overcome for MYO7A, responsible for USH1B, by using an equine infectious anemia virus (EIAV) based lentiviral vector, which has which has a cloning capacity of 9 kb (40).

However, the large size of the coding sequence of USH2A (15,606 bp; Genbank NM_206933) makes even EIAV-mediated transfer inaccessible for this gene.

Gene correction using genome-editing strategies, such as the CRISPR/Cas9 system, is a promising alternative for the treatment of IRDs. Genome editing allows targeted correction of disease-causing mutations instead of gene replacement (4, 7 and 11).

The CRISPR/Cas9 system is a bacterial adaptive immune system (8, 14 and 20) which has been largely used for in vivo and ex vivo genome editing therapies (31, 39 and 41). The system comprises two primary elements: the Cas9 nuclease and the single guide RNA molecule (gRNA). The Cas9 will induce a double strand break (DSB) at a specific locus in the DNA driven by the gRNA sequence and the protospacer adjacent motif (PAM), a three-nucleotide sequence found at the 3′ end of the gRNA sequence (NGG in the case of SpCas9) (17). After cleavage of the DNA, the target locus will typically undergo one of the two major pathways for DNA repair (25). In the error-prone non-homologous end joining (NHEJ) pathway, the two ends of the DSB are randomly re-ligated, leaving insertions and deletions (INDELs) at the desired region (3). Alternatively, homology-directed repair (HDR) using a donor template can be used to precisely edit the genome at the desired region (8).

The coupling of the CRISPR/Cas9 system together with patient-specific induced pluripotent stem cell (iPSC) technology has opened up a window for regenerative therapy and personalized medicine for patients. The correction of the patient's own cells could lead to the generation of autologous iPSC-derived retinal cells for transplantation therapy. In this way, patients could avoid immunosuppressive regimes without the risk of graft rejection (21).

However, to the inventors' knowledge, no studies have demonstrated efficient correction of both the c.2276G>T and c.2299delG mutations in the USH2A genes.

Therefore, there remains a need to develop a novel gene therapy strategy to efficiently treat patients suffering from either Usher syndrome type 2 or arRP presenting one of the two aforementioned USH2A mutations.

### SUMMARY OF THE INVENTION

The Applicant identified a method which addressed the aforementioned needs.

A first object of the present invention accordingly relates to an in vitro or ex vivo method for correcting the two most prevalent USH2A mutations.

In particular, a first object of the present invention accordingly relates to an in vitro or ex vivo method for correcting at least one of the USH2A mutations, selected among c.2276G>T and c.2299delG mutations, both in exon 13, in the genome of an individual's induced pluripotent stem cells (iPSC), comprising the steps of:

(i) providing to the cell a site-directed genome-editing system by:
(a) providing to the said cell at least one guide nucleic acid (gRNA) comprising at least one nucleic acid sequence selected from the group consisting of SEQ ID NO: 1, SEQ ID NO:2 and SEQ ID NO: 7;
(b) providing to the said cell at least one Clustered regularly interspaced short palindromic repeats (CRISPR) associated nuclease, in particular at least one CRISPR associated protein 9 (Cas9), in particular at least one high efficiency CRISPR associated protein 9 (eSpCas9 (1.1)); and
(c) further providing to the said cell at least one donor nucleic acid that serves as a repair template for the mutated USH2A gene, in particular in the form of a single-stranded oligodeoxynucleic acid (ssODN);

(ii) culturing the cells obtained at step (i) such that the said at least one donor nucleic acid is integrated in the cell genome so as to correct at least one of the two most prevalent USH2A mutations.

In a particular embodiment, the induced pluripotent stem cell (iPSC) is derived from an in vitro processing of a cell previously collected from an individual having a genome bearing one or both of the USH2A gene mutations.

In particular, said individual is an individual suffering from inherited retinal dystrophies, in particular suffering from retinitis pigmentosa, more particularly suffering from isolated retinitis pigmentosa or retinitis pigmentosa in association with hearing loss as part of Usher syndrome type 2.

Another object of the invention relates to a genetically modified induced pluripotent cell (iPSC), in particular an iPSC wherein the c.2276G>T or the c.2299delG mutation has been corrected, obtainable by a method according to the invention as defined above.

Another object of the invention relates to a genetically modified induced pluripotent cell (iPSC), in particular an iPSC wherein the c.2276G>T mutation has been corrected, obtainable by a method according to the invention as defined above.

Another object relates to a genetically modified induced pluripotent stem cell (iPSC) wherein the c.2276G>T mutation and the c.2299delG mutation have been corrected, obtainable by a method according to the invention as defined above.

A further object of the invention relates to a pharmaceutical composition comprising at least one cell differentiated from a genetically modified iPSC of the invention in a pharmaceutically acceptable medium.

Another object of the invention relates to a genetically modified cell or a pharmaceutical composition according to the invention for their use as a medicament.

A further object of the invention relates to a genetically modified cell or a pharmaceutical composition according to the invention, for use in the treatment of inherited retinal dystrophies, in particular suffering from retinitis pigmentosa, more particularly suffering from either isolated retinitis pigmentosa or retinitis pigmentosa in association with hearing loss as part of Usher syndrome type 2.

Another object of the invention relates to a site-directed genetic engineering system for correcting one or more USH2A gene mutations in the genome of a cell, such as a photoreceptor cell, of an individual in need thereof, comprising:

(i) at least one guide nucleic acid comprising at least one nucleic acid sequence selected from the group consisting of SEQ ID NO: 1, SEQ ID NO:2 and SEQ ID NO: 7;

(ii) at least one Clustered regularly interspaced short palindromic repeats (CRISPR) associated nuclease, in particular at least one CRISPR associated protein 9 (Cas9), in particular at least one high efficiency CRISPR associated protein 9 (eSpCas9(1.1));

(iii) at least one donor nucleic acid that serves as a repair template for the USH2A gene, in particular in the form of a single-stranded oligodeoxynucleic acid (ssODN), and

(iv) optionally at least one delivery vehicle comprising at least the elements of (i), (ii) and (iii).

In a particular embodiment, an individual in need thereof is an individual suffering from inherited retinal dystrophies, in particular suffering from retinitis pigmentosa, more particularly suffering from either isolated retinitis pigmentosa or retinitis pigmentosa in association with hearing loss as part of Usher syndrome type 2

### DESCRIPTION OF THE FIGURES

FIG. 1: Design of CRISPR/Cas9 strategy for correcting the most prevalent USH2A mutations in exon 13

A. Representation of exon 13 of the USH2A gene comprising the two most prevalent mutations (c.2276G>T and c.2299delG first and second arrows from left to right respectively), both present in exon 13 and located 22 bp from each other. This representation further indicates the locus in exon 13 of USH2A that is targeted by gRNA 1, gRNA 2, gRNA 3 and gRNA 4 which were designed by the inventors according to the presence of the canonical NGG PAM site, which is a requirement for eSpCas9 recognition.

B. Agarose gel electrophoresis results from the T7E1 assay after amplification for 30 cycles of the target locus of each gRNA “with an extra G” in the gRNA sequence.

Lanes: (from left to right) C− negative control (−: without T7E1, +: with T7E1); gRNA 1 (−: without T7E1, +: with T7E1); gRNA 2 (−: without T7E1, +: with T7E1), gRNA 3 (−: without T7E1, +: with T7E1), gRNA 4 (−: without T7E1, +: with T7E1), and a molecular weight ladder (bp) (MW).

C. Agarose gel electrophoresis results from the T7E1 assay after amplification for 30 cycles of the target locus of each gRNA with specific primers analyzed using agarose gel electrophoresis without the “extra G” in the gRNA sequence.

Lanes: (from left to right) C− negative control (−: without T7E1, +: with T7E1); gRNA 1 (−: without T7E1, +: with T7E1); gRNA 2 (−: without T7E1, +: with T7E1), gRNA 3 (−: without T7E1, +: with T7E1), gRNA 4 (−: without T7E1, +: with T7E1), gRNA 1 with an extra G in Cas9 WT plasmid (pX458) (−: without T7E1, +: with T7E1), gRNA 2 with an extra G (−: without T7E1, +: with T7E1), and a molecular weight ladder (bp) (MW).

D. Representation of single-stranded oligonucleotides ssODN 1 and ssODN 2 designed for the correction of the two mutations by homologous directed repaired (HDR). The ssODNs were designed using the reference sequence for USH2A. The ssODNs are complementary to the non-targeted strand by the gRNA. Silent changes of the PAM sequence were incorporated in the template to prevent Cas9 from re-cleavage after HDR. For ssODN 1, the PAM silent mutation destroys a NcoI site present in the reference sequence of USH2A. For ssODN the PAM silent mutation incorporates an MscI site.

FIG. 2: CRISPR/Cas9 mediated correction of the c.2299delG mutation in patient's iPSC

A. Agarose gel electrophoresis results after PCR amplification of the target region and restriction enzyme digestion from gDNA extracted from the 5 surviving clones analyzed.

Lanes: (from left to right) C− negative control, B1F11 clone, B3B8 clone, B3B1 clone, B2H4 clone, B2A3 clone and a molecular weight ladder (bp) (MW).

B. Confirmation of the HDR events by Sanger sequencing and cloning of the PCR products of FIG. 2A for each of the analyzed clones. Graph on the left: clone B1F11, graph on the right: clones B3B8, B3B1 and B2H4.

C. Heterozygous correction of B1F11 by the presence of the PAM silent mutation and the correction of the c.2299delG mutation in one of the alleles (A1), contrarily to the other allele (A2) in which Cas9-induced INDELs were observed (above).

Homozygous correction of B3B8, B3B1 and B2H4 of the c.2299delG mutation as well as homozygous introduction of the PAM silent mutation (below)

D. Confirmation of homozygous correction of B3B8, B3B1 and B2H4 and heterozygous correction of B1F11 by real-time quantitative PCR (qPCR) assay results presenting the copy number variation (CNV) of USH2A in the gDNA of the corrected clones. Graph on the left: the results were normalized to the housekeeping gene TERT; graph on the right: the results were normalized to the housekeeping gene TRMT10C.

Abscissa: (from left to right for each graph) C− negative control, B1F11 clone, B3B8 clone, B3B1 clone, and B2H4 clone.

Ordinate: copy number variation (CNV) normalization to TERT or TRMT10C.

FIG. 3: CRISPR/Cas9 mediated correction of the c.2276G>T mutation in patient's iPSC

A. Agarose gel electrophoresis results after PCR of the target region and NcoI digestion from gDNA extracted from 14 of the surviving clones analyzed.

Lanes: (from left to right) C− negative control, M1B4 clone, M1C7 clone, M1E3 clone, M3H10 clone, M2E1 clone, M1C2 clone, M2F10 clone, M3D11 clone, M1F4 clone, M2D3 clone, M1F11 clone, M2H6 clone, M3G7 clone, M3G5 clone and a molecular weight ladder (bp) (MW).

B. Cas9-induced modifications with gRNA 1 after cloning and sequencing of the 14 clones. From the top to the bottom: partial edition of allele 2 (A2) (40 clones out of 68 concerned), distribution of INDELs in A2 (22 clones out of 68 concerned), and allele 1 (A1) used to repair A2 (5 clones out of 68 concerned).

C. Representation of both alleles of exon 13 of the USH2A gene comprising the two most prevalent mutations (c.2276G>T and c.2299delG, second and third arrows from left to right, respectively), and the identified SNP (rs111033281, first arrow from the left).

D. Representation of both alleles of exon 13 of the USH2A gene comprising the two most prevalent mutations (c.2276G>T and c.2299delG second and third arrows from left to right respectively), and further indicating the localization that is targeted by gRNA 1 and gRNA 1S, which incorporates the identified SNP (rs111033281, first arrow from the left) and thus recognizes the missense variant allele A1 of exon 13.

E. Distribution results of Cas9-induced modification in A1 of exon 13 and of Cas9-induced modifications in A2 or in both A1 and A2 of exon 13 after nucleofecting eSpCas9 (1.1)-gRNA 1S together with ssODN 1 into the USH2A-USH-iPSC cell line.

F. Examples of the Cas9-induced modifications with gRNA 1S after cloning and sequencing for some of the nucleofected clones. From the top to the bottom: partial repair in A1 and A2 (2 clones out of 36 concerned), partial repair in A1 (5 clones out of 36 concerned), A2 was used to repair A1 (5 clones out of 36 concerned), distribution of INDELs in A1 (10 clones out of 36 concerned), distribution of INDELs in A2 (8 clones out of 36 concerned), positive clone MS3F7 for the correction of the missense variant (1 clone out of 36 concerned).

G. Confirmation of the HDR events by Sanger sequencing and cloning of the PCR products for positive clone MS3F7 and confirmation of the correction of the missense variant.

FIG. 4: Characterization of CRISPR/Cas9 corrected iPSC

A. Microscope observation of the CRISPR corrected iPSC clones USH2A-USH-iPSC-B3B1 (homozygous correction of c.2299delG) (above) and USH2A-RP-iPSC-MS3F7 (heterozygous correction of c.2276G>T) (below) demonstrating that the gene targeting process did not affect the pluripotency characteristics of their parental lines.

B. Genomic integrity of the CRISPR corrected iPSC clones USH2A-USH-iPSC-B3B1 (homozygous correction of c.2299delG) (above) and USH2A-RP-iPSC-MS3F7 (heterozygous correction of c.2276G>T) (below) as assessed by the iCS-digital TM Pluri test, which detects over 90% of the recurrent abnormalities in human pluripotent stem cells.

Ordinate: copy number of each chromosome.

Abscissa: Chromosomes identified by their usual designation.

C. Immunofluorescence analysis of the CRISPR iPSC clones USH2A-USH-iPSC-B3B1 (homozygous correction of c.2299delG) (above) and USH2A-RP-iPSC-MS3F7 (heterozygous correction of c.2276G>T) (below) by observation of their ability to retain expression of typical pluripotency markers OCT3/4, SOX2 and NANOG (from left to right).

D. Microscope observation of an embryoid body (EB) assay to determine the ability of CRISPR-corrected iPSC clones USH2A-USH-iPSC-B3B1 (homozygous correction of c.2299delG) (above) and USH2A-RP-iPSC-MS3F7 (heterozygous correction of c.2276G>T) (below) to differentiate into the three germ layers as assessed by immunostaining of (from left to right) Hepatocyte Nuclear Factor 4 (HNF4a) for endoderm, Smooth Muscle Actin (SMA) for mesoderm and Glial Fibrillary Acidic Protein (GFAP) for ectoderm (from left to right).

FIG. 5: Evaluating the mRNA expression levels of USH2A in CRISPR corrected iPSC

A. Evaluation of mRNA levels of restored USH2A by qPCR in exon 39 (left) and exon 13 (left) in CRISPR corrected iPSC clones USH2A-USH-iPSC-B3B1 (homozygous correction of c.2299delG) compared to mRNA levels in wild type iPSCs and in USH2A iPSCs that have not been nucleofected.

Abscissa: (from left to right) WT iPSC, USH2A-USH-iPSC and USH2A-USH-iPSC clone B3B1

Ordinate: relative expression levels of mRNA.

B. Evaluation of mRNA levels of restored USH2A by qPCR in exon 39 (left) and exon 13 (left) in CRISPR corrected iPSC clones USH2A-RP-iPSC-MS3F7 (heterozygous correction of c.2276G>T) compared to mRNA levels in wild type iPSCs and in USH2A iPSCs that have not been nucleofected.

Abscissa: (from left to right) WT iPSC, USH2A-USH-iPSC and USH2A-USH-iPSC clone MS3F7.

Ordinate: relative expression levels of mRNA.

FIG. 6: Microscopic observation of the c.2299delG correction in USH2A retinal organoids

This figure represents a microscopic observation of retinal organoids (upper figure) to study and characterize defects associated with the c.2299delG mutation and Usher syndrome type 2. A zoom of a specific zone, namely the brush border, of the outer part of the organoid is further presented (lower figure).

From left to right: WT iPSC-derived organoid, USH2A-USH-iPSC-derived organoid and CRISPR/Cas9-corrected USH2A-USH-iPSC-derived organoid.

The microscope used for image acquisition was an Olympus CKX53. In the upper figure, the WT iPSC-derived organoid image was acquired with a 4× objective. The USH2A-USH-iPSC line organoid was acquired with a 4× objective. The CRISPR/Cas9-corrected USH2A-USH-iPSC-derived organoid was acquired with a 10× objective. The scale bars in the panel indicate 400 μm, 400 μm and 200 μm, respectively. In the lower figure, the WT iPSC-derived organoid image was acquired with a 20× objective. The USH2A-USH-iPSC-derived organoid was acquired with the 10× objective. The CRISPR/Cas9-corrected USH2A-USH-iPSC-derived organoid was acquired with the 10× objective.

FIG. 7: Microscopic observation of the c.2276G<T correction in USH2A retinal organoids

The figure is a microscopic observation of retinal organoids (upper figure) to study and characterize possible defects associated with the c.2276G>T mutation and retinitis pigmentosa (RP). A zoom of a specific zone, namely the brush border, of the outer part of the organoid is further presented (lower figure).

From left to right: WT iPSC-derived organoid, USH2A-RP-iPSC-derived organoid, and CRISPR/Cas9-corrected USH2A-RP-iPSC-derived organoid.

The microscope used for image acquisition was an Olympus CKX53. In the upper figure, the WT iPSC-derived organoid image was acquired with a 10× objective. The USH2A-RP-iPSC-derived organoid was acquired with a 10× objective. The CRISPR/Cas9-corrected USH2A-RP-iPSC-derived organoid was acquired with a 10× objective. The scale bars in all the panels indicate 150m. In the lower figure, the WT iPSC-derived organoid image was acquired with a 20× objective. The USH2A-RP-iPSC-derived organoid was acquired with a 20× objective. The CRISPR/Cas9-corrected USH2A-RP-iPSC-derived organoid was acquired with a 20× objective. The scale bars in all the panels indicate 50m.

### DETAILED DESCRIPTION OF THE INVENTION

The inventors managed to successfully correct the two most prevalent USH2A mutations in the genome of a cell, in particular in the genome of an induced pluripotent stem cell (iPSC), more particularly in the genome of an iPSC from two different individuals. As mentioned above, the two most prevalent USH2A gene mutations c.2276G>T (or p.Cys759Phe) and c.2299delG (or p.Glu767Serfs*21).

The first patient (homozygous mutation c.2299delG) presenting with Usher syndrome type 2 and the second patient (compound heterozygote for c.2276G>T and c.2299delG) presenting with arRP.

As demonstrated in the experimental part below, the inventors achieved a high efficiency rate of correction of the most prevalent mutation for USH2A (c.2299delG) in an iPSC cell line from a patient presenting USH2 syndrome and were able to correct the c.2276G>T mutation in the iPSCs of another patient presenting arRP.

Moreover, the generated USH2A gene corrected iPSCs displayed typical iPSC characteristics.

Finally, the inventors demonstrated that the differentiation of corrected iPSC cells according to the invention for both the c.2276G>T and c.2299delG mutations into retinal organoids provided organoids with a well-defined laminated structure, and a brush border similar to healthy retinal organoids. The non-corrected retinal organoids had a less well-defined laminated structure with a shorter, or absent, brush border.

Treatment based on the administration of an iPSC according to the invention provides a new tool for cell therapy and gene therapy applicable to a large number of patients presenting with syndromic retinitis pigmentosa (Usher syndrome type 2) and/or isolated arRP, for which there is currently no treatment available.

Ex Vivo or In Vitro Method for Correcting One or More USH2A Gene Mutations

As indicated above, a first object of the present invention relates to an in vitro or ex vivo method for correcting at least one of the two most prevalent USH2A mutations.

In particular, a first object of the present invention relates to an in vitro or ex vivo method for correcting at least one of the two USH2A mutations selected among c.2276G>T and c.2299delG mutations, both in exon 13, in the genome of an individual's induced pluripotent stem cell (iPSC), comprising the steps of:

(i) providing to the cell a site-directed genetic engineering system by:
(a) providing to the said cell at least one guide nucleic acid (gRNA) comprising at least one nucleic acid sequence selected from the group consisting of SEQ ID NO: 1, SEQ ID NO:2 and SEQ ID NO: 7;
(b) providing to the said cell at least one Clustered regularly interspaced short palindromic repeats (CRISPR) associated nuclease, in particular at least one CRISPR associated protein 9 (Cas9), in particular at least one high efficiency CRISPR associated protein 9 (eSpCas9 (1.1)); and
(c) further providing to the said cell at least one donor nucleic acid that serves as a repair template for the mutated USH2A gene, in particular in the form of a single-stranded oligodeoxynucleic acid (ssODN);

(ii) culturing the cell obtained at step (i) such that the said at least one donor nucleic acid is integrated in the cell genome so as to correct the one or more USH2A gene mutations.

Induced pluripotent stem cells (iPSCs) are genetically reprogrammed adult cells that exhibit a pluripotent stem cell-like state similar to embryonic stem cells (ESCs). They are artificially generated stem cells that are not known to exist in the human body but show qualities similar to those of ESC. Generating such cells is well known in the art as discussed in Ying WANG et al. (47) as well as in Lapillonne H. et al. (48) and in J. DIAS et al. (49).

iPSCs are typically derived by introducing products of specific sets of pluripotency-associated genes, or “reprogramming factors”, into a given cell type, which are well known to one skilled in the art.

For instance, iPSCs may be generated from human fibroblasts.

The generation of iPSCs is crucially dependent on the transcription factors used for the induction.

Since iPSCs can be derived directly from adult tissues, they not only bypass the need for embryos, but can be made in a patient-matched manner, which means that each individual could have their own pluripotent stem cell line.

As such, in a particular embodiment, the iPSC according to the invention is derived from an in vitro processing of a cell previously collected from an individual having a genome bearing one or both of the two most prevalent USH2A gene mutations.

In a particular embodiment, the individual having a genome bearing one or more USH2A gene mutations is an individual suffering from inherited retinal dystrophies, in particular suffering from retinitis pigmentosa, and more particularly suffering from isolated retinitis pigmentosa or retinitis pigmentosa in association with hearing loss as part of Usher syndrome type 2.

iPSCs as described herein are preferably purified. The same applies for the ocular cells as defined below.

Many methods for purifying iPSCs are known in the art.

As used herein, “purified iPSCs” or “purified ocular cells” means that the recited cells make up at least 50% of the cells in a purified sample; more preferably at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the cells in a purified sample.

The cells selection and/or the cells purification can be performed by using both positive and negative selection methods to obtain a substantially pure population of cells.

In one aspect, fluorescence activated cell sorting (FACS), also referred to as flow cytometry, can be used to sort and analyze the different cell populations. Cells having the cellular markers specific for iPSC are tagged with an antibody, or typically a mixture of antibodies, that binds the cellular markers. Each antibody directed to a different marker is conjugated to a detectable molecule, particularly a fluorescent dye that can be distinguished from other fluorescent dyes coupled to other antibodies. A stream of stained cells is passed through a light source that excites the fluorochrome and the emission spectrum from the cells detects the presence of a particular labelled antibody. By concurrent detection of different fluorochromes, cells displaying different sets of cell markers are identified and isolated from other cells in the population. Other FACS parameters, including, by way of example and not limitation, side scatter (SSC), forward scatter (FSC), and vital dye staining (e.g., with propidium iodide) allow selection of cells based on size and viability.

In another aspect, immunomagnetic labelling can be used to sort the different cell population. This method is based on the attachment of small magnetizable particles to cells via antibodies or lectins. When the mixed population of cells is placed in a magnetic field, the cells that have beads attached will be attracted by the magnet and may thus be separated from the unlabeled cells.

In a particular embodiment, the cell previously collected and from which the iPSC is derived may be an autologous cell, i.e. a cell collected from the individual bearing one or more mutations in the USH2A gene, and to which subsequent administration of the cells corrected by the method disclosed herein is contemplated.

“Autologous” refers to deriving from or originating from the same patient or individual. An “autologous transplant” refers to the harvesting and reinfusion or transplant of a subject's own cells or organs. Exclusive or supplemental use of autologous cells can eliminate or reduce many adverse effects of administration of the cells back to the host, particular host reaction.

In this case, in a particular embodiment, the initial cell from which the iPSC is derived is previously collected from an individual having a genome bearing one or more USH2A gene mutations, in particular from an individual suffering from inherited retinal dystrophies, from inherited retinal dystrophies, in particular suffering from retinitis pigmentosa, and more particularly suffering from isolated retinitis pigmentosa or retinitis pigmentosa in association with hearing loss as part of Usher syndrome type 2.

In another embodiment, the initial population of iPSCs may be derived from an allogeneic donor or from a plurality of allogeneic donors. The donors may be related or unrelated to each other, and in the transplant setting, related or unrelated to the recipient (or individual).

As already specified herein, the genome of the cells, and in particular of the iPSCs, according to the invention as described herein, are genetically corrected for one or more USH2A gene mutations, the c.2276G>T and c.2299delG mutations.

The USH2A gene is an autosomal recessive gene located in chromosome 1 and comprising 72 exons.

The USH2A gene codes for a protein called usherin. Usherin is an important component of basement membranes, which are thin, sheet-like structures that separate and support cells in many tissues. Usherin is found in the sensory hair cells of the inner ear and in the light-sensing photoreceptors of the retina, which is the tissue lining the back of the eye. Although the function of usherin has not been well established, studies suggest that it is part of a group of proteins (a protein complex) that plays an important role in the development and maintenance of cells in the inner ear and retina.

As mentioned above, USH2A is the main gene responsible for inherited retinal dystrophies, in particular in individuals suffering from retinitis pigmentosa, and more particularly suffering from isolated retinitis pigmentosa or retinitis pigmentosa in association with hearing loss as part of Usher syndrome type 2). The two most prevalent mutations identified in USH2A, which are located 22 bp from each other in exon 13 are c.2276G>T (or p.Cys759Phe) and c.2299delG (or p.Glu767Serfs*21).

An individual suffering from USH2 may contain at least one copy of c.2299delG.

An individual suffering from autosomal recessive retinitis pigmentosa may contain at least one copy of c.2276G>T.

The mutation is qualified as homozygous if it is present on both alleles of the USH2A gene.

For example, a homozygous c.2299delG mutation of the USH2A gene means that both of the alleles of the USH2A gene contain the c.2299delG mutation.

On the contrary, the mutation is qualified as heterozygous if the mutation is different from one allele to the other.

For example, a heterozygous c.2299delG mutation of the USH2A gene means that one allele contains the c.2299delG mutation, while the other allele does not.

An individual suffering from arRP may contain heterozygous c.2299delG and c.2276G>T mutations of the USH2A gene.

Step (i) (a) of an in vitro or ex vivo method for correcting one or more USH2A gene mutations in the genome of a cell as described herein, is defined as providing to the said cell at least one guide nucleic acid (gRNA) comprising at least one nucleic acid sequence selected from the group consisting of SEQ ID NO: 1, SEQ ID NO:2 and SEQ ID NO: 7.

gRNAs are target-specific short single-stranded RNA sequences. Commonly referred as gRNA, it is the fusion of two RNA molecules: a CRISPR RNA (crRNA) and a trans-activating RNA (tracrRNA), which is equally effective in binding to target DNA.

gRNAs usually comprise an 80-nucleotide constant region and a short 20-nucleotide target-specific sequence (in 5′ of the gRNA sequence) that binds to a DNA target via Watson-Crick base pairing.

gRNAs are artificial and do not exist in nature.

The sequences of a gRNA, as indicated above, are RNA sequences that are complementary to their targeted DNA sequence.

The gRNA comprising at least one nucleic acid sequence of SEQ ID NO: 7 was designed by the inventors and cloned to incorporate a sequence complementary to a SNP (single nucleotide polymorphism) that was identified in the allele comprising the c.2276G>T mutation. The identified SNP is c.2256T>C in cis with c.2276G>T in A1. This gRNA is able to recognize the missense variant allele.

This SNP is present in the genome of 75% of patients carrying the c.2276G>T mutations of the USH2A gene. Therefore, the majority of the patients carrying the c.2276G>T allele also carry the SNP in the same allele.

The design of a gRNA comprising a sequence complementary to this identified SNP has greatly improved the recognition of the targeted allele.

In a particular embodiment, the at least one gRNA consists of at least one nucleic acid sequence selected from the group consisting of SEQ ID NO: 1, SEQ ID NO: 2 and SEQ ID NO: 7.

As defined above, step (i) (b) of a method as described herein is defined as providing to the said cell at least one Clustered regularly interspaced short palindromic repeats (CRISPR) associated nuclease, in particular at least one CRISPR associated protein 9 (Cas9), in particular at least one high efficiency CRISPR associated protein 9 (eSpCas9 (1.1)).

The CRISPR associated nuclease according to the invention is devoid of target site specificity, i.e. the said nuclease is not able to recognize by itself a specific target site in the genome of the cell described herein.

In order to specifically cleave DNA at a particular target site, such nuclease needs to be associated to a guide nucleic acid (gRNA), as described above, binding to the selected target site and inducing a double strand break (DSB).

According to the invention, and as mentioned above, the selected target site is located in exon 13 of the USH2A gene comprised in the genome of the said cell.

When guided to the target site by the guide nucleic acid (gRNA), a nuclease as described herein is able to introduce a double-stranded break in the said target site.

In a particular embodiment, the at least one CRISPR associated nuclease is at least one CRISPR associated protein 9 (Cas9), in particular is at least one high efficiency CRIPSR associated protein 9 (eSpCas9 (1.1)).

As defined above, step (i) (c) of a method as described herein is defined as providing to the said cell at least one donor nucleic acid that serves as a repair template for the mutated USH2A gene, in particular in the form of a single-stranded oligodeoxynucleic acid (ssODN).

Indeed, the aim of the method according to the invention is to correct at least one of the two most prevalent USH2A gene mutations in the genome of a cell.

Once the CRISPR associated Cas9 nuclease, in particular the eSpCas9 (1.1), has cleaved the DNA strands of the USH2A gene at the end of step (i) (b), the cell will use its natural ability to repair itself.

The presence of the at least one donor nucleic acid that serves as a repair template is to direct the cell towards an alternative repair pathway, i.e. towards homology-directed repair (HDR).

To accomplish this, the at least one donor nucleic acid that serves as a repair template, in particular in the form of ssODN, bears the desired sequence, which must be introduced in the genome of the cell. In the present case, the at least one donor nucleic acid that serves as a repair template bears the non-mutated USH2A gene.

A certain number of cells will use this template to repair the broken sequence via homologous recombination, thereby incorporating the desired corrections into the genome.

The at least one donor nucleic acid that serves as a repair template may be selected from the group consisting of the sister chromatid in the other allele of the cell, a exogenous plasmid/vector or a single-stranded oligonucleotides (ssODN). In a particular embodiment, the at least one donor nucleic acid that serves as a repair template is a single-stranded oligonucleotide (ssODN).

ssODNs have been shown to be effective and powerful templates for directing HDR upon DSB in the genome (Strouse, Bialk, Niamat, Rivera-torres, & Kmiec, 2014) (36). A previous study demonstrated that asymmetric ssODN complementary to the non-targeted strand enhance HDR.

In a particular embodiment, at least one donor nucleic acid that serves as a repair template, in particular a ssODN, is complementary to the strand not targeted by the gRNA.

In a particular embodiment, the donor nucleic acid that serves as a repair template, in particular a ssODN, is asymmetrical.

An asymmetrical or asymmetrical designed donor nucleic acid is a nucleic acid that comprises a different number of nucleotides on either side of the nuclease target site of the CRISPR associated Cas9 nuclease.

In particular, an asymmetrical ssODN suitable for the invention may be selected from asymmetrical ssODN comprising a proximal sequence (that is the sequence before the nuclease target site) containing 71 nucleotides and a distal sequence (that is the sequence after the nuclease target site) containing 49 nucleotides, asymmetrical ssODN comprising a proximal sequence containing 21 nucleotides and a distal sequence containing 36 nucleotides, asymmetrical ssODNs comprising a proximal sequence containing 21 nucleotides and a distal sequence containing 49 nucleotides, asymmetrical ssODNs comprising a proximal sequence containing 21 nucleotides and a distal sequence containing 89 nucleotides, asymmetrical ssODNs comprising a proximal sequence containing 71 nucleotides and a distal sequence containing 26 nucleotides, asymmetrical ssODNs comprising a proximal sequence containing 71 nucleotides and a distal sequence containing 16 nucleotides, asymmetrical ssODNs comprising a proximal sequence containing 71 nucleotides and a distal sequence containing 36 nucleotides, asymmetrical ssODNs comprising a proximal sequence containing 91 nucleotides and a distal sequence containing 36 nucleotides, or asymmetrical ssODNs comprising a proximal sequence containing 114 nucleotides and a distal sequence containing 83 nucleotides.

In a particular embodiment, an asymmetrical ssODN of the invention comprises a proximal sequence containing 91 nucleotides and a distal sequence containing 36 nucleotides.

For example, the ssODNs defined in the experimental part below (see Example 1) were asymmetrically designed and contained 91 nucleotides in the PAM-proximal region and 36 nucleotides in the PAM-distal region.

In a particular embodiment, the donor nucleic acid that serves as a repair template, in particular a ssODN, comprises, at one end, preferably at both ends (that is in 5′ and 3′), at least one modified terminal base. In particular, the donor nucleic acid that serves as a repair template, in particular a ssODN, comprises two modified terminal bases at each of the both ends.

Preferably, the donor nucleic acid that serves as a repair template, in particular a ssODN, comprises, at one end, preferably at both ends, at least one phosphorothioate-modified terminal base. In particular, the donor nucleic acid that serves as a repair template, in particular a ssODN, comprises two phosphorothioate-modified terminal bases at each of the both ends.

These modified terminal bases allow to enhance the stability of the donor nucleic acid(s).

Preferably, the modifications are present in the two first nucleotides of the sequence and in the two last nucleotides of the sequence of the donor nucleic acid that serves as a repair template, in particular a ssODN.

In a preferred embodiment, the at least one donor nucleic acid is at least one ssODN, complementary to the strand non-targeted by the gRNA, that is asymmetrical, and comprising at one end, preferably at both ends, at least one modified terminal base, in particular comprising at least one phosphorothioate-modified terminal base, preferably two, at each of the both ends.

The use of this particular design of ssODN has been shown to increase mutation correction mediated by HDR. In addition, the modifications added at both ends of the ssODN help stabilization of the ssODN to perform HDR events.

In a particular embodiment, the at least one donor nucleic acid that serves as a repair template for the mutated USH2A gene is designed using the reference sequence for USH2A (15,606 bp; Genbank NM_206933).

In a particular embodiment, the at least one donor nucleic acid that serves as a repair template comprises part of the exon 13 of the USH2A gene.

In particular, the at least one donor nucleic acid that serves as a repair template comprises at least the part of the sequence of exon 13 of the USH2A gene that comprises one or two of the mutation sites.

In a particular embodiment, the at least one donor nucleic acid that serves as a repair template comprises at least one nucleic acid sequence selected from the group consisting of SEQ ID NO: 5 and SEQ ID NO: 6. In a particular embodiment, the at least one donor nucleic acid that serves as a repair template consists of at least one nucleic acid sequence is selected from the group consisting of SEQ ID NO: 5 and SEQ ID NO: 6.

Steps (i)(a), (i)(b) and (i)(c) of the method as described herein can be independently realized simultaneously or separately from one another. In a preferred embodiment, the at least one guide nucleic acid (gRNA) of step (i)(a), the at least one Clustered regularly interspaced short palindromic repeats (CRISPR) associated nuclease, in particular eSpCas9 (1.1), of step (i)(b) and at least one donor nucleic acid that serves as a repair template for the mutated USH2A gene of step (i)(c) are provided simultaneously to the said cell.

Methods to introduce in vitro, ex vivo or in vivo proteins and nucleic acid molecules into cells are well known in the art. The traditional methods to introduce a nucleic acid, usually present in a vector, or a protein in a cell include microinjection, electroporation and sonoporation. Other techniques based on physical, mechanical and biochemical approaches such as magnetofection, optoinjection, optoporation, optical transfection and laserfection can also be mentioned (42).

A Clustered regularly interspaced short palindromic repeats (CRISPR) associated protein, (Cas) as described herein is in particular the CRISPR associated protein 9 (Cas9).

In a particular embodiment, the at least one Clustered regularly interspaced short palindromic repeats (CRISPR) associated nuclease is a high efficiency CRISPR associated protein 9 (Cas9), in particular is eSpCas9 (1.1).

eSpCas9 (1.1) is an enhanced specificity Cas 9 nuclease which harbors K848A/K1003A/R1060A mutations and which was generated to decrease protein affinity for the non-target DNA strand, thereby decreasing the stability of mismatch-containing helices (43). It has shown to assess DNA cleavage in human cells with significant reduction in off-targets but maintaining a robust on-target activity.

CRISPR-Cas systems for genome editing are particular systems using simple base pairing rules between an engineered RNA and the target DNA site instead of other systems using protein-DNA interactions for targeting.

CRISPR-Cas RNA-guided nucleases are derived from an adaptive immune system that evolved in bacteria to defend against invading plasmids and viruses.

According to an embodiment, it consists of a mechanism by which short sequences of invading nucleic acids are incorporated into CRISPR loci. They are then transcribed and processed into CRISPR RNAs (crRNAs) which, together with a trans-activating crRNAs (tracrRNAs), complex with CRISPR-associated (Cas) proteins to dictate specificity of DNA cleavage by Cas nucleases through Watson-Crick base pairing between nucleic acids. The crRNA harbors a variable sequence known as the “protospacer” sequence. The protospacer-encoded portion of the crRNA directs Cas9 to cleave complementary target DNA sequences if they are adjacent to short sequences known as “protospacer adjacent motifs” (PAMs). Protospacer sequences incorporated into the CRISPR are not cleaved because they are not present next to a PAM sequence (see 44 and 45).

According to this embodiment, a guide RNA (gRNA) as described herein corresponds to the fusion of the crRNA and tracrRNA, which is known as gRNA. The term guide RNA or gRNA used in the present text designates this particular form.

According to one embodiment, a method of the invention may implement at least one guide nucleic acid (gRNA) comprising at least one nucleic acid sequence selected from the group consisting of SEQ ID NO: 1, SEQ ID NO:2 and SEQ ID NO: 7, and preferably is SEQ ID NO: 7, at least one high efficiency CRISPR associated protein 9, preferably eSpCas9 (1.1), and an asymmetrical single-stranded oligodeoxynucleic acid (ssODN), preferably comprising at one end, preferably at both ends, at least one, and preferably two, modified terminal bases.

As defined above, step (ii) of a method as described herein is defined as culturing the cell obtained at step (i) such that the said at least one donor nucleic acid is integrated in the cell genome so as to correct the one or more USH2A gene mutations.

As such, at the end of step (ii), the genome of the cell contains a corrected USH2A gene, which means that the USH2A gene can be transcribed into a functional mRNA and further translated into a functional protein.

A functional mRNA is an mRNA whose activity is at least equal to the activity of an mRNA transcribed from an USH2A gene which does not contain any mutations.

A functional protein is a protein whose activity is at least equal to the activity of a protein translated from an mRNA that was transcribed from an USH2A gene, which does not contain any mutations.

Genetically Modified Induced Pluripotent Stem Cell

The present invention also relates to a genetically modified induced pluripotent stem cell (iPSC), obtainable by a method according to the invention as defined above.

In particular, the invention relates to a genetically modified induced pluripotent stem cell (iPSC) wherein the c.2276G>T mutation and/or the c.2299delG mutation have been corrected, obtainable by a method according to the invention as defined above.

In particular, the invention relates to a genetically modified induced pluripotent stem cell (iPSC) wherein the c.2276G>T mutation has been corrected, obtainable by a method according to the invention as defined above.

In particular, the invention relates to a genetically modified induced pluripotent stem cell (iPSC) wherein the c.2276G>T mutation and the c.2299delG mutation have been corrected, obtainable by a method according to the invention as defined above.

In some embodiments, a system for correcting one or more USH2A gene mutations as disclosed herein, comprising (i) one or more guide nucleic acid (gRNA), (ii) at least one CRISPR associated nuclease, in particular at least one CRISPR associated protein 9 (Cas9), in particular at least one high efficiency CRISPR associated protein 9, eSpCas9 (1.1), (iii) at least one donor nucleic acid, in particular in the form of an ssODN and (iv) optionally at least one delivery vehicle comprising the elements (i), (ii) and (iii), may be suitable for local administration to an individual in need thereof, i.e. to an individual having a genome bearing one or more mutations in the USH2A gene. Local administration of the said system to the said individual in need thereof will lead to administration of the said system to one or more cells of the said individual in need thereof.

Treatment based on the administration of cell differentiated from an iPSC obtainable according to the invention may be used in cell therapy for treating a large number of patients suffering from inherited retinal dystrophies, in particular suffering from retinitis pigmentosa, and more particularly suffering from isolated retinitis pigmentosa or retinitis pigmentosa in association with hearing loss as part of Usher syndrome type 2).

Pharmaceutical Composition

The present invention also relates to a pharmaceutical composition comprising at least one cell differentiated from a genetically modified iPSC according to the invention as defined above, in a pharmaceutically acceptable medium.

A pharmaceutically acceptable medium as described herein is in particular suitable for administration to a mammalian individual.

A “pharmaceutically acceptable medium” comprises any of standard pharmaceutically accepted mediums known to those of ordinary skill in the art in formulating pharmaceutical compositions, in particular in formulating pharmaceutical compositions to be administered to the eye.

According to the invention, at least one cell differentiated from a genetically modified iPSC according to the invention is a cell which is obtained after culturing a genetically modified iPSC as prepared according to the invention until it has differentiated into a particular cell. The culture and cell differentiation is done under appropriate conditions and includes one or more lineage-specific differentiation factors.

These differentiation factors are well known to one skilled in the art and are selected according to the end-cell that is needed.

In particular, the at least one cell differentiated from a genetically modified iPSC according to the invention is at least one photoreceptor cell or a photoreceptor precursor.

Photoreceptor cells are light-sensitive ocular cells.

There are currently three known types of photoreceptor cells in mammalian eyes: rods, cones, and intrinsically photosensitive retinal ganglion cells. The two classic photoreceptor cells are rods and cones, each contributing information used by the visual system to form a representation of the visual world, sight. The rods are narrower than the cones and distributed differently across the retina, but the chemical process in each that supports phototransduction is similar. A third class of mammalian photoreceptor cell was discovered during the 1990s: the intrinsically photosensitive retinal ganglion cells.

In a particular embodiment, the at least one cell differentiated from a genetically modified iPSC according to the invention is a rod precursor cell.

In an embodiment, the cells as described herein can be used in a composition in combination with other cells as defined above, but not modified as described herein.

In an embodiment, the cells as described herein can be used in a composition in combination with other agents and compounds that enhance the therapeutic effect of the administered cells.

In another embodiment, the cells as described herein can be administered in a composition with therapeutic compounds that enhance the differentiation of the cells as described herein. These therapeutic compounds have the effect of inducing differentiation and mobilization of the cells that are endogenous, and/or the ones that are administered to the individual as part of the therapy.

Genetically Modified Induced Pluripotent Stem Cells (iPSC) for their Use as a Medicament

Another object of the present invention is a genetically modified cell according to the invention or a pharmaceutical composition according to the invention, for its use as a medicament.

In order to be used as a medicament, the genetically modified induced pluripotent stem cells (iPSC) according to the invention should be cultured into a particular differentiated cell.

To obtain a differentiated cell that can be used in a medicament according to the invention, the genetically-modified induced pluripotent stem cells (iPSCs) should be cultured under appropriate conditions. The culture medium should include one or more lineage-specific differentiation factors. These differentiation factors are well known to one skilled in the art and are selected according to the end-cell that is needed.

In particular, a differentiated cell obtained from a genetically modified iPSC according to the invention is at least one photoreceptor cell or a photoreceptor precursor.

Cells according to the invention can be administered by well-known methods. Cells as described herein are best suited for local administration, in particular for subretinal administration.

The number of cells needed for achieving a therapeutic effect will be determined empirically in accordance with conventional procedures for the particular purpose.

Generally, for administering the cells for therapeutic purposes, the cells are given at a pharmacologically effective dose.

By “pharmacologically effective amount” or “pharmacologically effective dose” is meant an amount sufficient to produce the desired physiological effect or amount capable of achieving the desired result, particularly for treating the disorder or disease condition, including reducing or eliminating one or more symptoms or manifestations of the disorder or disease.

Illustratively, administration of cells to a patient suffering from Usher syndrome provides a therapeutic benefit when the amount of usherin protein coded by USH2A gene in the patient is increased, when compared to the amount of usherin protein in the patient before administration.

The number of cells transfused will take into consideration factors such as sex, age, weight, the types of disease or disorder, stage of the disorder, the percentage of the desired cells in the cell population (e.g., purity of cell population), and the cell number needed to produce a therapeutic benefit.

A pharmaceutical composition as described herein, as previously mentioned, can be used for administration of the cells as described herein into the individual in need thereof.

The administration of cells can be through a single administration or successive administrations. When successive administrations are involved, different cells numbers and/or different cells populations may be used for each administration.

Illustratively, a first administration can be of a cell or a cell population as described herein that provides an immediate therapeutic benefit as well as more prolonged effect, while the second administration includes cells as described herein that provide prolonged effect to extend the therapeutic effect of the first administration.

A further object of the invention relates to a genetically modified cell according to the invention or a pharmaceutical composition according to the invention for use in the treatment of inherited retinal dystrophies, in particular of retinitis pigmentosa, more particularly of isolated retinitis pigmentosa or retinitis pigmentosa in association with hearing loss as part of Usher syndrome type 2.

It can also be mentioned a method for the treatment of inherited retinal dystrophies, in particular of retinitis pigmentosa, more particularly of isolated retinitis pigmentosa or retinitis pigmentosa in association with hearing loss as part of Usher syndrome type 2 in an individual in need thereof comprising the administration of a genetically modified cell according to the invention and/or a pharmaceutical composition according to the invention as described herein to an individual in need thereof.

It can also be mentioned the use of a genetically modified cell according to the invention as described herein for the manufacture of a medicament for treating inherited retinal dystrophies, in particular retinitis pigmentosa, more particularly isolated retinitis pigmentosa or retinitis pigmentosa in association with hearing loss as part of Usher syndrome type 2 in an individual in need thereof.

A Site-Directed Genetic Engineering System for Correcting One or More USH2A Gene Mutations

The present invention also relates to a site-directed genetic engineering system for correcting at least one of the two most prevalent USH2A gene mutations.

In particular, the present invention also relates to a site-directed genetic engineering system for correcting at least one of the two USH2A gene mutations, selected among c.2276G>T and c.2299delG mutations, in the genome of a cell, such as of a photoreceptor cell, of an individual in need thereof, comprising:

(i) at least one guide nucleic acid comprising at least one nucleic acid sequence (gRNA) selected from the group consisting of SEQ ID NO: 1, SEQ ID NO:2 and SEQ ID NO: 7;

(ii) at least one Clustered regularly interspaced short palindromic repeats (CRISPR) associated nuclease, in particular at least one CRISPR associated protein 9 (Cas9), in particular at least one high efficiency CRISPR associated protein 9 (eSpCas9(1.1));

(iii) at least one donor nucleic acid that serves as a repair template for the USH2A gene, in particular in the form of a single-stranded oligodeoxynucleic acid (ssODN), and

(iv) optionally at least one delivery vehicle comprising at least the elements of (i), (ii) and (iii).

Treatment based on the administration of a system according to the invention and described herein may be used in gene therapy for treating a large number of patients suffering from inherited retinal dystrophies in particular suffering from retinitis pigmentosa, more particularly suffering from isolated retinitis pigmentosa or retinitis pigmentosa in association with hearing loss as part of Usher syndrome type 2, for which there is currently no treatment available.

The elements of (i), (ii) and (iii) constituting a system according to the invention can be as previously detailed in the present text.

As mentioned above, inherited retinal dystrophies (IRD) caused by mutations in the USH2A gene may severely affect the eye and its individual cells.

As such, the system for correcting one or more USH2A gene mutations is directed in particular to the genome of an ocular cell, in particular of a photoreceptor cell.

In a particular embodiment, the system for correcting one or more USH2A gene mutations in the genome of a cell, notably of a photoreceptor cell, may further optionally comprise at least one delivery vehicle comprising at least the elements of (i), (ii) and (iii).

When the system according to the invention is used as part of a gene therapy treatment, the delivery vehicle may be used to administrate the different elements of the system to the individual to be treated.

In a particular embodiment, the at least one delivery vehicle is selected from the group consisting of viral vectors and non-viral vectors.

Viral vectors are successful gene therapy systems such as retrovirus, adenovirus (types 2 and 5), adeno-associated virus (AAV), herpes virus, pox virus, human foamy virus (HFV), and lentivirus. All viral vector genomes have been modified by deleting some areas of their genomes so that their replication becomes deranged and it makes them safer to administrate to a patient. During the past few years, some viral vectors with specific receptors have been designed that could transfer the transgenes to some other specific cells, which are not their natural target cells (retargeting).

In some embodiments, one skilled in the art will prefer to use more than one viral vector as the delivery vehicles for the elements (i), (ii) and/or (iii). These viral vectors may be identical or different.

In a particular embodiment, the at least one delivery vehicle is at least one viral vector. In particular, the at least one viral vector is selected from the group consisting of retroviral vectors, adenoviral vectors, adeno-associated virus vectors, herpes simplex virus vectors, lentivectors, poxvirus vectors and Epstein-Barr virus vectors, and in particular is selected from adeno-associated virus vectors.

Non-viral vectors mainly comprise chemical systems that are not of viral origin and generally include chemical methods such as cationic liposomes and polymers. Efficiency of these vectors may sometimes be less than viral systems in gene transduction, but their cost-effectiveness, availability, and more importantly less induction of immune system and no limitation in size of transgenic DNA compared with viral systems have made them more effective for gene delivery.

Viral and non-viral vectors that may be used according to the invention are well known to the skilled in the art, and are, for example, described in Nayerossadat et al. (50).

Alternatively, in another embodiment, the elements of (i), (ii) and (iii) of the system according to the invention may be administered to the individual to be treated through other means, without the need for a delivery vehicle.

A further object of the present invention relates to a system according to the invention and described herein for use in the treatment of inherited retinal dystrophies, in particular in the treatment of inherited retinal dystrophies, in particular in the treatment of retinitis pigmentosa, more particularly of isolated retinitis pigmentosa or retinitis pigmentosa associated with hearing loss as part of Usher syndrome type 2.

When the system according to the invention is used in the treatment of inherited retinal dystrophies, it may in particular be administrated in the form of a pharmaceutical composition further comprising a pharmaceutically acceptable medium.

The pharmaceutical composition comprising the system for correcting at least one of the two most prevalent USH2A gene mutations, the c.2276G>T and c.2299delG mutations, in the genome of a cell, notably of a photoreceptor cell, according to the invention and the pharmaceutically acceptable medium can be as previously detailed in the present text.

In accordance, one skilled in the art understands that the system for correcting at least one of the two most prevalent USH2A gene mutations according to the invention cannot be used directly to prepare a pharmaceutical composition, but will serve as a platform to prepare such a composition.

Indeed, it is the genetically modified cell, in particular the genetically modified photoreceptor cell precursor, obtained from using the system according to the invention, whose genome has been corrected, which will serve to prepare a pharmaceutical composition.

In a particular embodiment, the pharmaceutical composition is suitable for a local administration to the individual to be treated, such as is suitable for an administration to the eye of the individual to be treated.

Ocular drug delivery has been a major challenge to pharmacologists and drug delivery scientists due to its unique anatomy and physiology. Static barriers (different layers of cornea, sclera, and retina including blood aqueous and blood-retinal barriers), dynamic barriers (choroidal and conjunctival blood flow, lymphatic clearance, and tear dilution), and efflux pumps in conjunction pose a significant challenge for delivery of a drug alone or in a dosage form, especially to the posterior segment.

The three primary methods of delivery of pharmaceutical compositions to the eye are topical, local ocular (i.e. subconjunctival, intravitreal, retrobulbar, intracameral), and systemic. Each one of these methods has its benefits and its challenges. As such, the pharmaceutical composition comprising the system according to the invention should be adapted to these methods of delivery.

The most appropriate method of administration depends on the area of the eye to be treated. The administration form and the pharmaceutically acceptable medium according to the invention thus also need to be suitable for administration to the area of the eye to be treated.

For example, a system for correcting one or more USH2A gene mutations in the genome of a photoreceptor cell according to the invention may be suitable for subretinal administration. To date, subretinal delivery has been widely applied by scientists and clinicians as a more precise and efficient route of ocular drug delivery for gene therapies and cell therapies including stem cells in diseases such as retinitis pigmentosa.

In particular, subretinal injection has more direct effects on the targeting cells in the subretinal space.

These ocular administration forms are well known to the skilled and the art and are described, for example, in Peng et al. (51).

The present invention is further illustrated by, without in any way being limited to, the examples herein.

### EXAMPLES

### Example 1: Design of CRISPR/Cas9 Strategy for Correcting the Most Prevalent USH2A Mutations in Exon 13

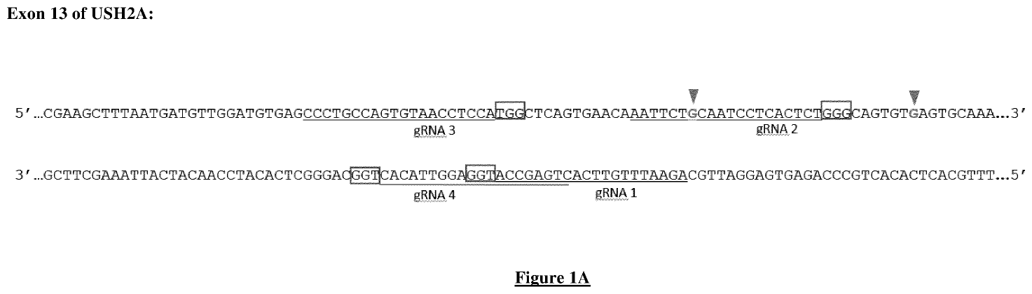

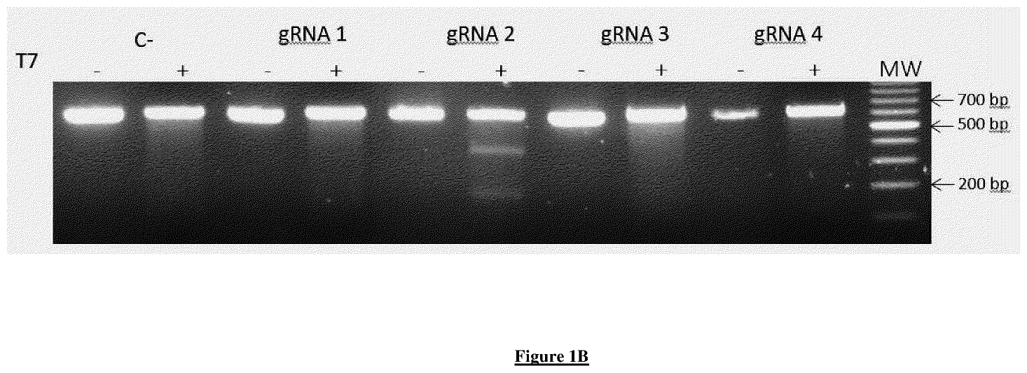

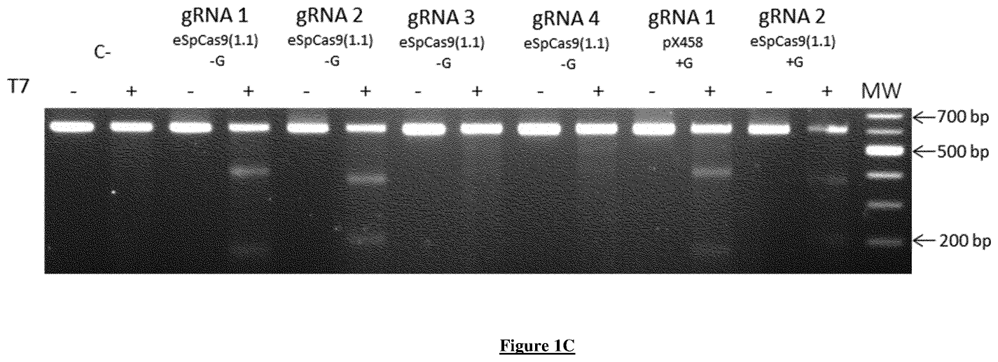

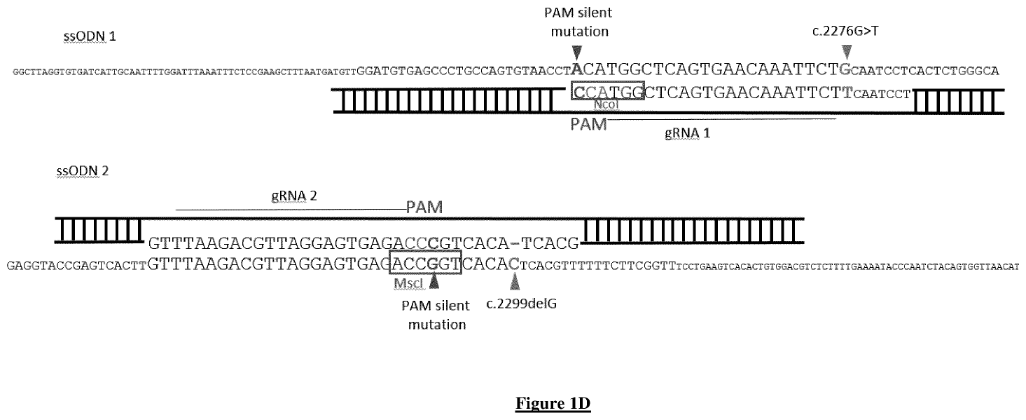

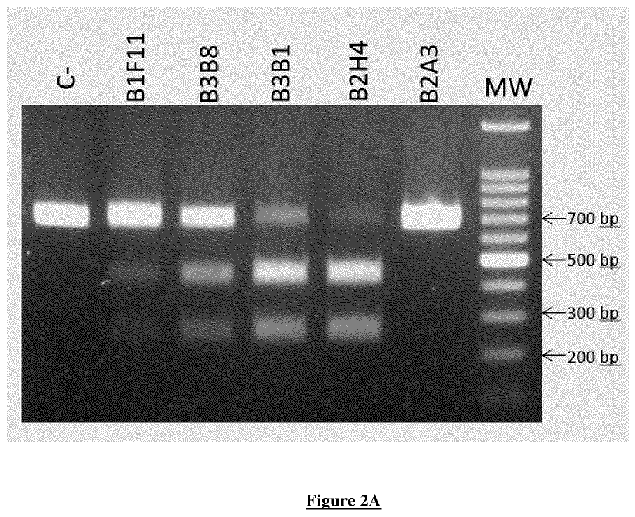

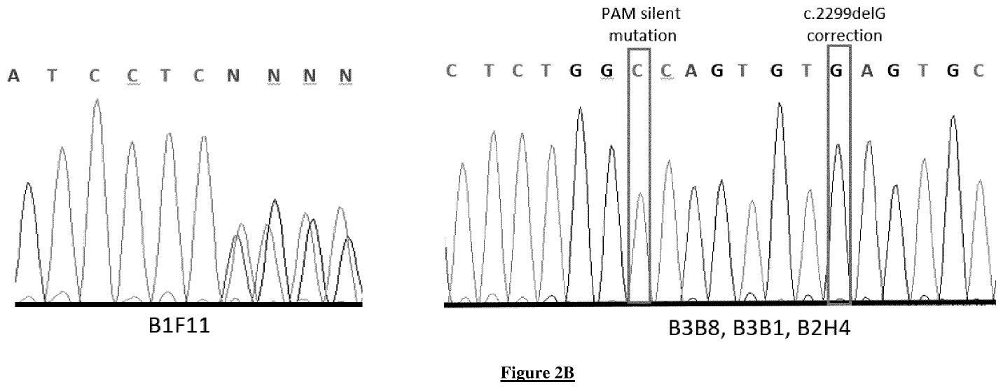

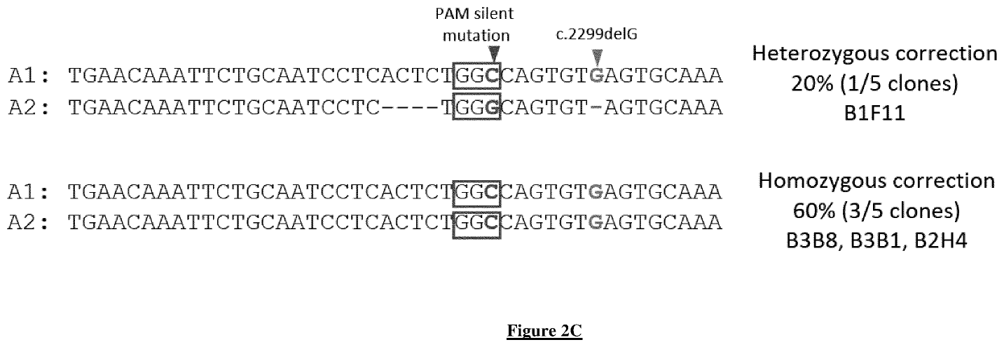

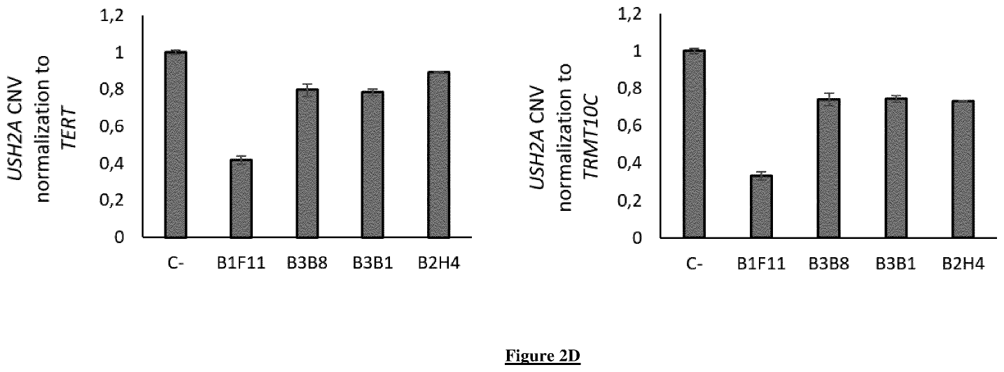

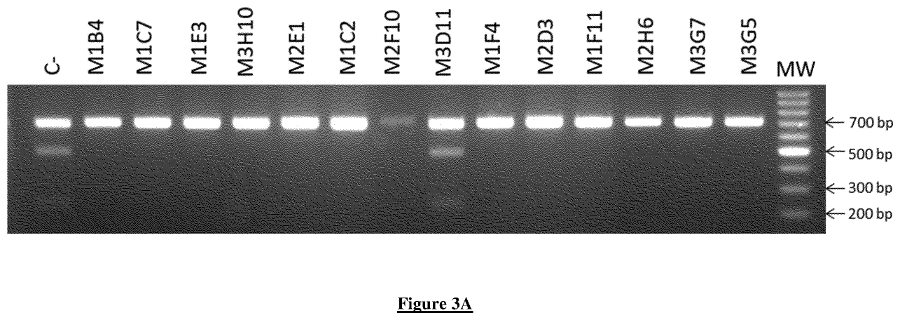

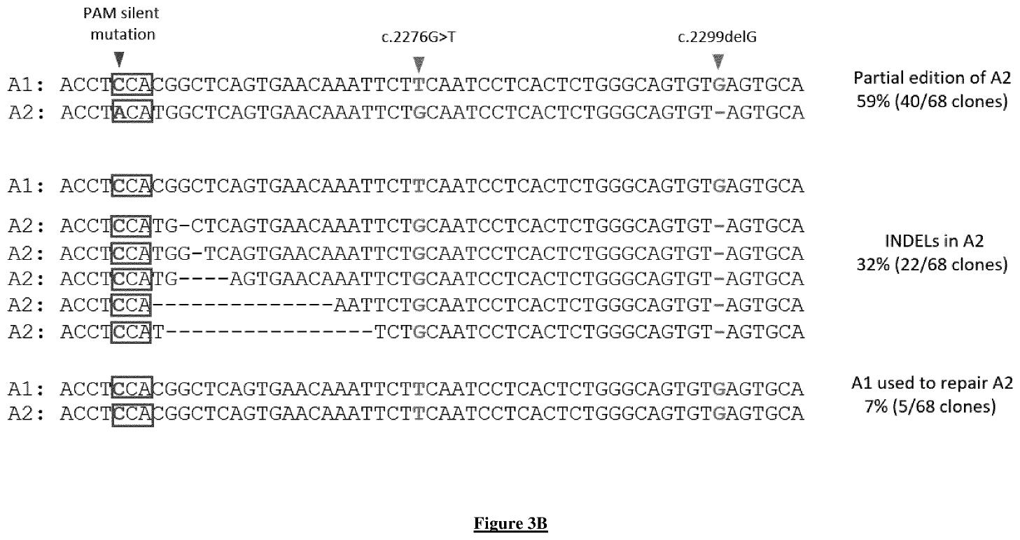

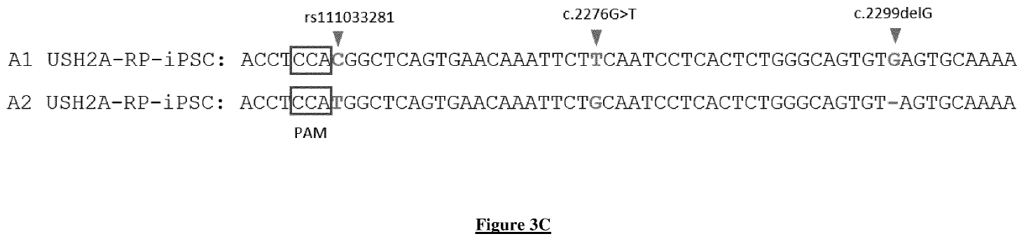

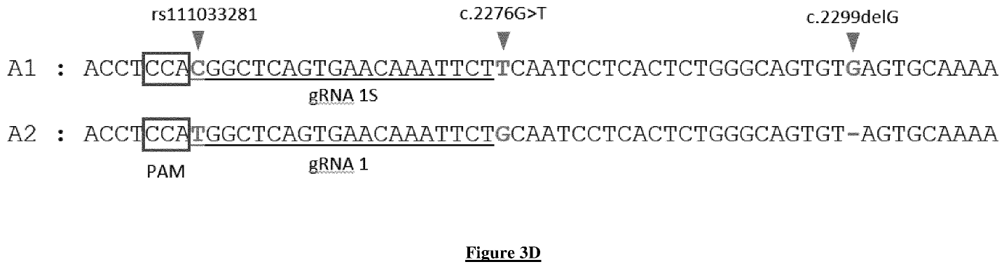

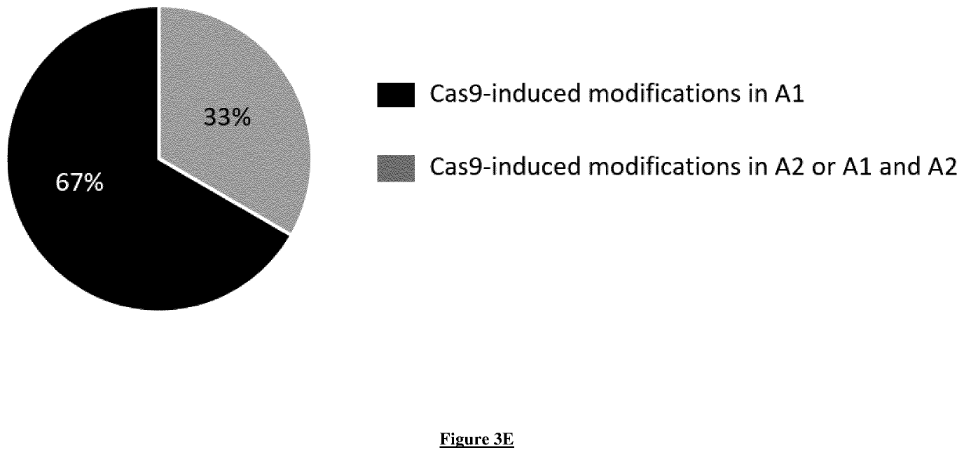

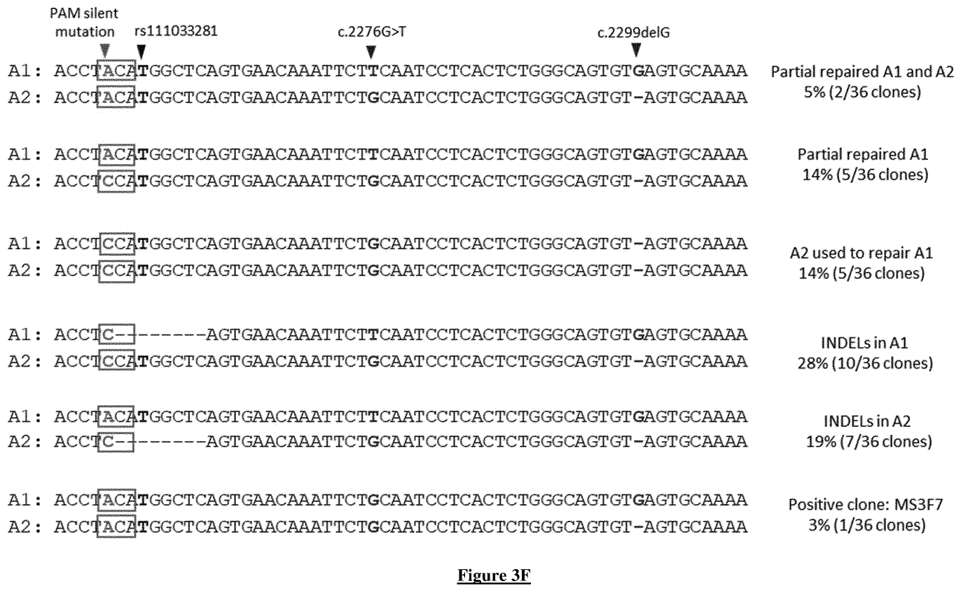

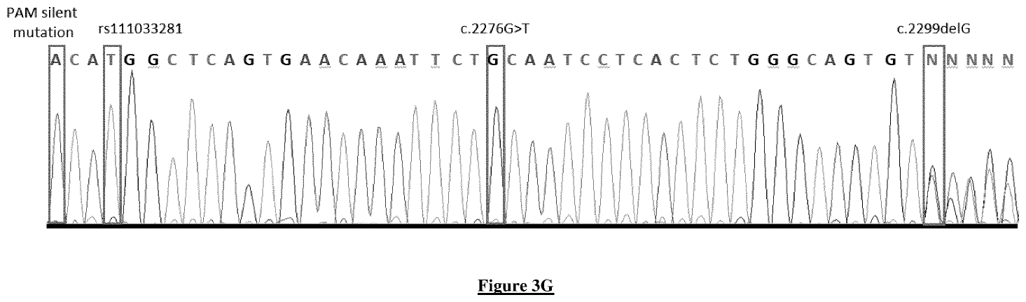

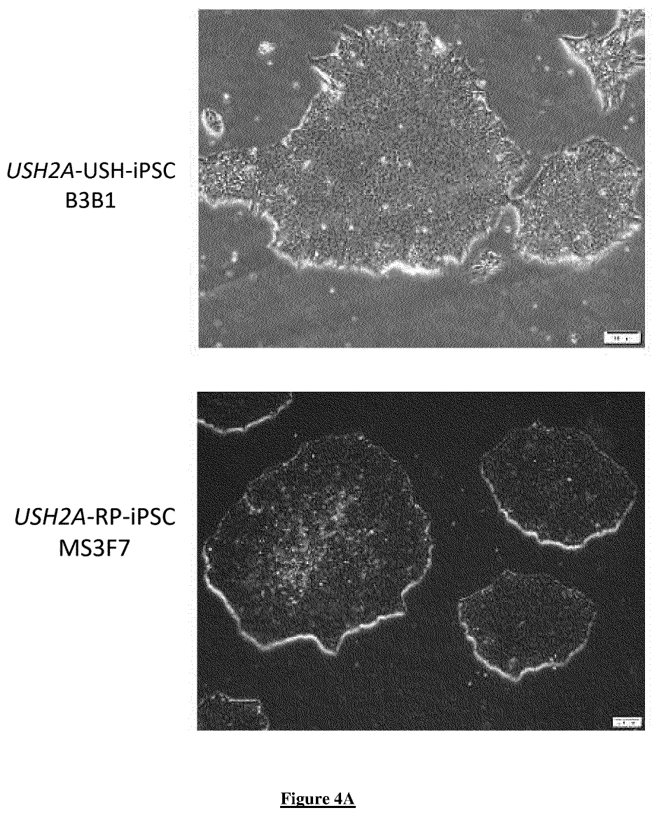

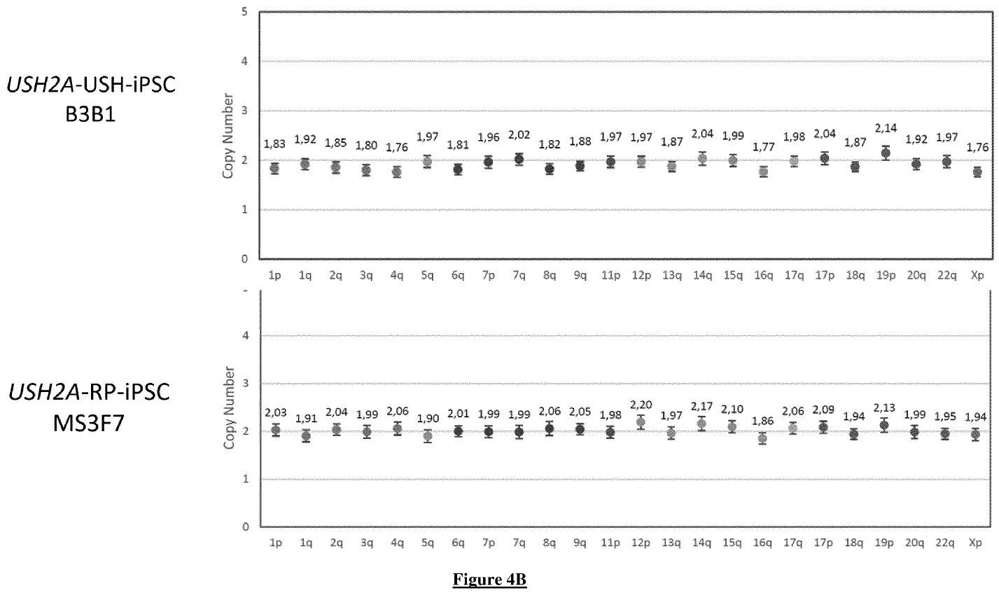

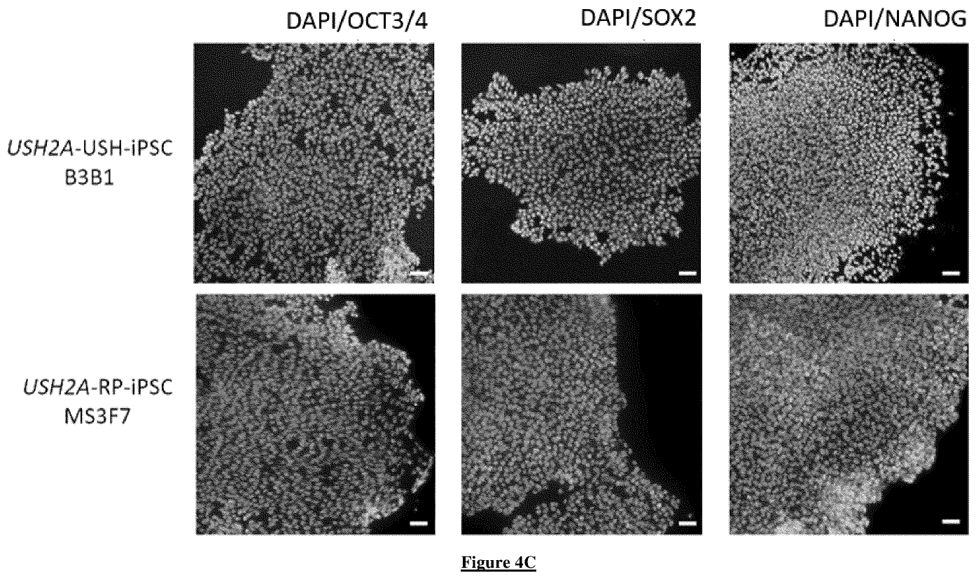

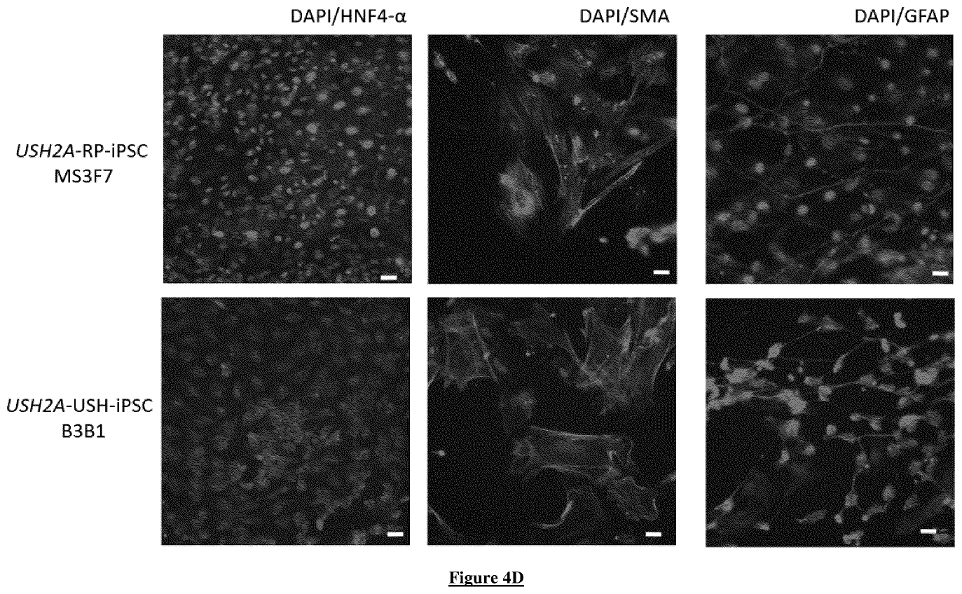

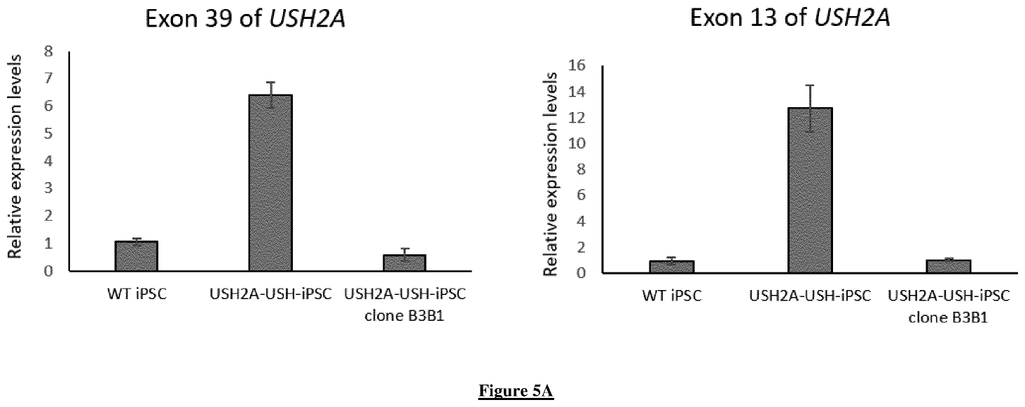

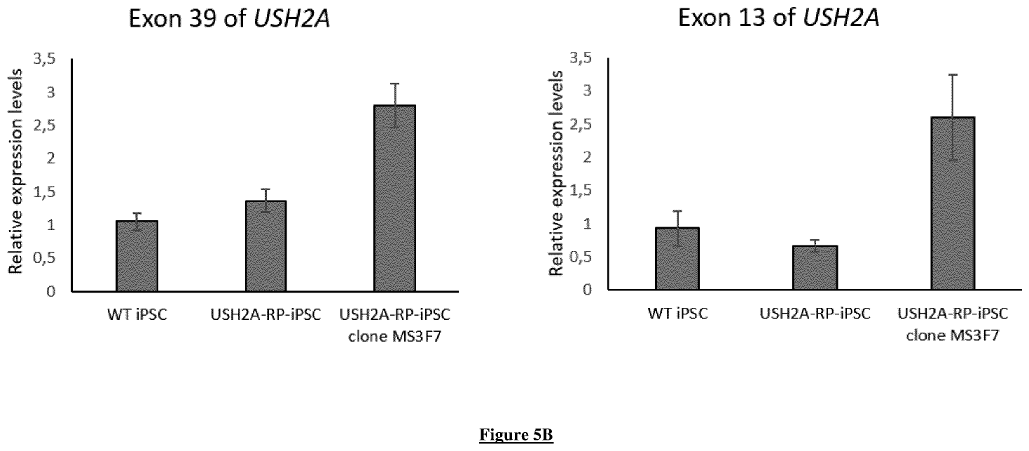

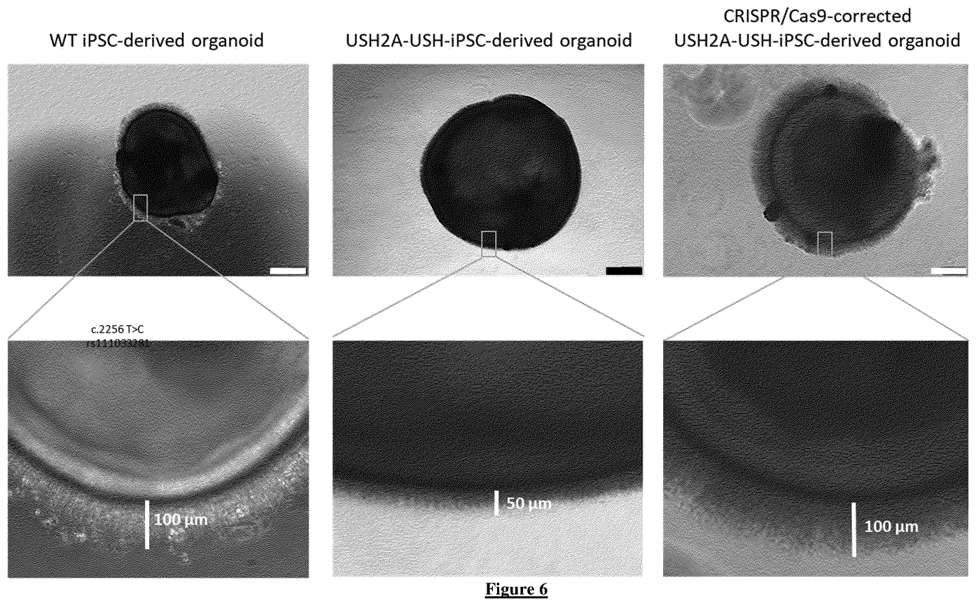

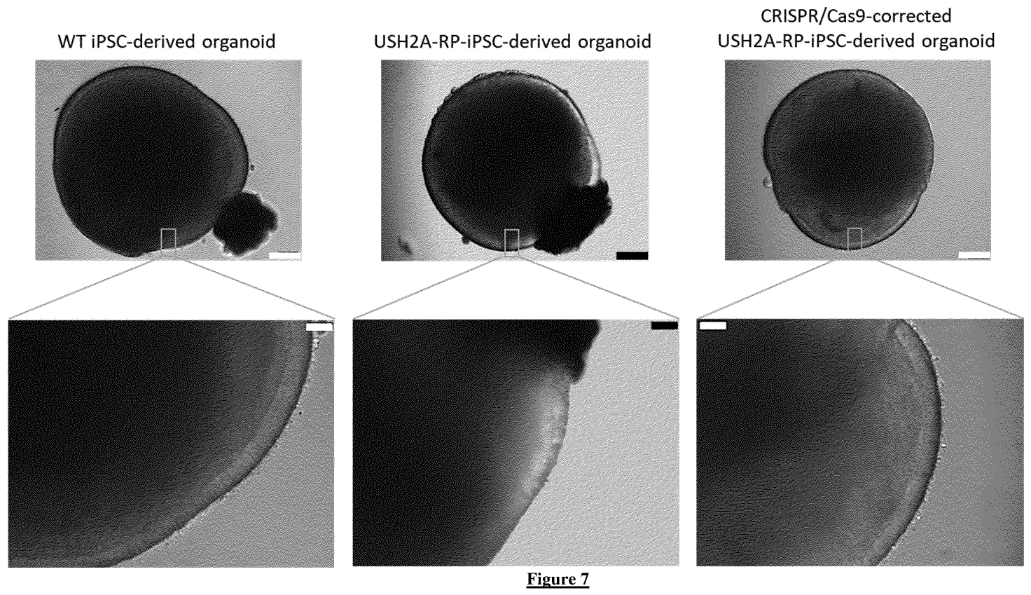

In the present study, the inventors aimed to correct the two most prevalent mutations in USH2A (c.2276G>T and c.2299delG), both present in exon 13 and located 22 bp from each other (FIG. 1A).

Four different gRNAs surrounding both mutations were designed (gRNA 1-4) according to the presence of the canonical NGG PAM site, which is a requirement for SpCas9 recognition: gRNA 1 (SEQ ID NO: 1), gRNA 2 (SEQ ID NO: 2), gRNA 3 (SEQ ID NO: 3) and gRNA 4 (SEQ ID NO: 4) (FIG. 1A).

All four gRNAs were cloned into the “enhanced specificity” Cas9 plasmid (eSpCas9 (1.1), Addgene #71814). This plasmid co-expresses the gRNA and the eSpCas9 (1.1) with EGFP, which is linked to the C-terminal of eSpCas9 by a 2A peptide. This variant of the wild type Cas9 has been shown to induce DNA cleavage in human cells with significant reduction in off-targets, while maintaining a robust on-target activity (34). Because the expression of the gRNA in the eSpCas9 (1.1) plasmid is driven by the human U6 promoter that “prefers” a G to start transcription (27), all four gRNAs were designed and cloned with an extra “G” at the 5′ of the gRNA sequence.

To determine the cleavage efficiency of the selected gRNAs, eSpCas9 (1.1) plasmids containing the gRNAs were individually transfected into HEK293 cells using Lipofectamine 3000.

HEK293 cells were maintained in DMEM/F12 (Gibco) supplemented with 10% fetal bovine serum (FBS) (Gibco) and 1% penicillin-streptomycin (PS) (Gibco).

For the validation of the gRNAs 3×10⁵ HEK293 cells were transfected separately with 1.5 μg of each of the plasmid constructs using Lipofectamine 3000 (Invitrogen) according to the manufacture's recommendations. Forty-eight hours after transfection, cells were harvested for genomic DNA extraction.

The ability of the four gRNAs to induce cleavage at the target site was assessed by the T7-endonuclease I (T7E1) assay. The target locus was amplified for 30 cycles with specific targeting exon 13 of USH2A using high fidelity LA TAKARA polymerase. The PCR product was then denatured by heating at 95° C. for 5 minutes and reannealed by the following program: ramp down to 85° C. at −2° C./s, and ramp down to 25° C. at −0.1° C./s. After, 0.50 of T7 endonuclease 1 (New England Bioloabs) was added to the mix and incubated at 37° C. for 15 minutes. The reaction was stopped by adding 1 μl of proteinase K and incubating the mix for 5 minutes at 37° C. The digested product was analyzed using agarose gel electrophoresis.

The results from the T7E1 assay demonstrated that only one gRNA (gRNA 2) was able to induce a DSB in the DNA (FIG. 1B).

Recent studies have suggested that the presence of the extra “G” in the gRNA when using an “enhanced specificity” Cas9 might interfere with the on-target Cas9 activity (Kato-inui, Takahashi, Hsu, & Miyaoka, 2018) (15). For this reason, all four gRNAs were re-designed without the extra “G” and cloned into the eSpCas9 (1.1) plasmid. The new plasmids containing the gRNAs without the extra “G”, were transfected into HEK293 and a T7E1 assay was run to assess their activity.

The T7E1 assay demonstrated cleavage activity for gRNA 1 and again for gRNA 2, even though there was no detectable activity for gRNA 3 and gRNA 4 (FIG. 1C).

To confirm that the cleavage activity of gRNA 1 was due to the presence or absence of the extra “G” in the eSpCas9 (1.1) plasmid, the inventors cloned gRNA 1 with the extra “G” into a wild type Cas9 plasmid (pX458, Addgene #106097) and re-performed a T7E1 assay on transfected HEK293 cells.

In view of the results, gRNA 1 and gRNA 2 both without the extra “G” were selected for the correction of the missense variant c.2276G>T and the c.2299delG mutation respectively.

These results demonstrate the gRNAs 1 and 2 present a higher recognition efficiency that those of gRNAs 3 and 4 and are thus better suited according to the invention.

After validation of the activity of the two gRNAs in HEK293 cells, repair templates in form of single-stranded oligonucleotides (ssODN) were designed for the correction of the two mutations by HDR.

A previous study demonstrated that asymmetric ssODN complementary to the non-targeted strand enhance HDR (29), therefore the ssODNs were designed following this criteria and using the reference sequence for USH2A (15,606 bp; Genbank NM_206933).

The ssODNs were asymmetrically designed containing 91 nucleotides in the PAM-proximal region and 36 nucleotides in the PAM-distal region according to (29). In addition they were designed complementary to the non-targeted strand by the gRNA. Silent changes of the PAM sequence were incorporated in the template to prevent Cas9 from re-cleavage after HDR. The ssODNs were purchased from GENEWIZ with phosphorothioate modifications to enhance HDR (29).

Following this method, two ssODNs were designed: ssODN 1 (SEQ ID NO: 5), for gRNA 1, in which the change for the correction of the c.2276G>T mutation was introduced and ssODN 2 (SEQ ID NO: 6) for gRNA 2, to correct the c.2299delG mutation (FIG. 1D).

In addition, to avoid re-cleavage of the target DNA by the Cas9 after HDR, PAM silent mutations were introduced in both ssODN sequences (24). For ssODN 1 the PAM silent mutation destroyed an NcoI restriction enzyme site present in the USH2A reference sequence. Contrarily, the PAM silent mutation for ssODN 2 introduced an MscI site at the targeted sequence. These two PAM silent mutations facilitated genotyping of HDR events. To enhance ssODN stability phosphorothioate-modified terminal bases at both ends of the ssODNs were added.

### Example 2: CRISPR/Cas9 Mediated Correction of the c.2299delG Mutation in Patient's iPSC

Aiming at correcting the most prevalent USH2A mutation in patient's iPSC, an iPSC cell line (USH2A-USH-iPSC) from a patient presenting USH2 syndrome due to the homozygous mutation c.2299delG was used with this purpose (32).

First, a skin biopsy from the patient was performed under sterile conditions at the Centre of Reference for Genetic Sensory Disorders (CHRU Montpellier) following informed consent. Regional and national ethic committees accorded biomedical research approval under the authorisation number 2014-A00549-38. Human fibroblasts were cultured in AmnioMAX C100 basal medium supplemented with 10% decomplemented FCS (Lonza, Verviers, Belgium), 1% GlutaMAX (Gibco, ThermoFisher Scientific, Villebon sur Yvette, France), 1% penicillin-streptomycin-amphotericin B (Lonza) and 2% AmnioMax-C100 supplement (Gibco) at 37° C. under 5% CO2.

Next, to generate integration-free iPSCs, the fibroblasts cells from the corresponding patient were transduced with the CytoTune-iPS 2.0 Sendai reprogramming kit (Life Technologies) containing three Sendai virus-based reprogramming vectors (CytoTune 2.0-KOS, -hc-MYC, -hKLF4) expressing KLF4, OCT4, SOX2, and/or c-MYC, according to the manufacturer's recommendations. The following medium: high glucose DMEM containing GlutaMAX (Gibco) and supplemented with 10% FBS (Gibco), 1% non-essential amino acids (Gibco) and 55 mM b-mercaptoethanol (Gibco) was refreshed daily for 7 days. On day 7 the transduced fibroblasts were plated onto Matrigel-coated culture dishes. On day 8, the media was changed to TeSR-E7 Basal Medium (Stemcell Technologies, Grenoble, France). From day 15, emerging iPSC colonies were mechanically passaged using a scalpel and cultured in Essential 8 (E8) medium (Gibco) under 5% CO2 at 37° C. The culture medium was refreshed daily, and cells were passaged twice a week using Versene (Gibco).

The USH2A-USH-iPSC cell line obtained was nucleofected with the eSpCas9 (1.1)-gRNA 2 plasmid in combination with ssODN 2. Forty-eight hours post-nucleofection, iPSCs were GFP single-cell sorted by fluorescence-activated cell sorting (FACS) and re-seeded in 96 well-plates. The surviving iPSC colonies (5 out of 288) were then expanded for further culture, characterization and screening of HDR events.

Due to the PAM silent mutation introduced in ssODN 2, which creates an MscI site, the inventors first screened the clones for HDR events by PCR-amplification of the target region in extracted DNA and restriction enzyme digestion. MscI digestion of the PCR product showed that 4 out of the 5 surviving clones had used ssODN 2 to repair the Cas9-induced DSB (FIG. 2A).

Confirmation of the HDR events was achieved by Sanger sequencing and cloning of the PCR products (FIG. 2B).

Of the four positive clones analyzed, one clone (B1F11) showed heterozygous correction, as determined by the presence of the PAM silent mutation and the corrected c.2299delG mutation in one allele (A1), compared to the second allele (A2) in which Cas9-induced INDELs were observed (FIG. 2C). The remaining three positive clones (B3B8, B3B1 and B2H4) demonstrated homozygous correction of the c.2299delG mutation as well as homozygous introduction of the PAM silent mutation (FIG. 2C).

To rule out any large Cas9-induced deletions (1) in exon 13 of USH2A, preventing PCR amplification of both alleles, the inventors designed a real-time quantitative PCR (qPCR) assay to estimate the copy number variation (CNV) of USH2A in the gDNA of the corrected clones (D'haene, Vandesompele, & Hellemans, 2010) (9).

RNA was isolated using the QiaShredder and RNeasy mini kits (Qiagen) according to the manufacturer's instructions. The isolated RNA was treated with RNase-Free DNase (Qiagen) and 0.5 μg was reverse transcribed using the Superscript III Reverse Transcriptase kit (Life Technologies). For the CNV studies 250 ng of gDNA were used for the experiment and results were normalized to TERT or TRMT10C. For the USH2A expression studies, 5 ng of cDNA were used per reaction and results were normalized to GAPDH. Reactions were performed using the LightCycler®480 SYBR Green I Master mix on a LightCycler®480 II thermal cycler (Roche). Results were analyzed using LightCyclerV 480 software and the Microsoft Excel program.

A primer set was designed surrounding the c.2299delG mutational site. The forward primer was directly designed to hybridize in the region where B1F11 clone presented INDELs in A2.

The qPCR results showed that all the homozygous clones (B3B8, B3B1 and B2H4) presented similar CNV relative values of USH2A compared to the negative control (C−, USH2A-USH-iPSC clone without CRISPR/Cas9-induced genome editing). By contrast, the heterozygous-corrected B1F11 clone presented half the relative CNV values of USH2A (FIG. 2D).

Taking all of these results together, the inventors achieved a high efficiency rate (4 out of 5 survival clones) of correction of the most prevalent mutation for USH2A in an iPSC cell line from a patient presenting USH2 syndrome.

### Example 3: CRISPR/Cas9 Mediated Correction of the c.2276G>T Mutation in Patient's iPSC

To correct the USH2A most prevalent mutation in autosomal recessive retinitis pigmentosa (arRP), the inventors used an iPSC cell line (USH2A-RP-iPSC) from a patient presenting non-syndromic RP due to a compound heterozygous mutation (c.2276G>T and c.2299delG) (31).

This cell line was prepared following the same protocol as the one described in example 2, namely by a performing a skin biopsy from the patient, followed by culture of the fibroblasts and generating non-integrating iPSCs.

In this cell line, the inventors aimed at correcting the hypomorfic c.2276G>T missense variant by using the already validated gRNA 1 in combination with ssODN 1. For this purpose, USH2A-RP-iPSC cell line was nucleofected with the eSpCas9 (1.1)-gRNA 1 and ssODN 1 and GFP-single cell sorted was carried out 48 hours after nucleofection.

By contrast to the USH2A-USH-iPSC cell line, 68 clones (out of 288) from the USH2A-RP-iPSC cell line survived. The surviving clones were then amplified and initially screened for HDR events by NcoI digestion of the PCR-amplified target region. A representative gel of 14 NcoI digested clones is shown in FIG. 3A. Encouragingly, all of the clones, with the exception of one (M3D11), seemed to have incorporated the ssODN 1 into the host DNA as they could not be cut by NcoI.

In light of the promising results, the inventors sequenced all the 68 survival clones. Surprisingly, the sequencing results were not consistent with the restriction enzyme digestion results. Initial sequencing analysis suggested that for all 68 clones only the non-target allele, allele 2 (A2), had been modified. These results were further confirmed by sub-cloning and re-sequencing of the target region (FIG. 3B).

In 59% (40/68) of the clones, the PAM silent mutation was only introduced in A2. In 32% (22/68) of the clones, we detected Cas9-induced INDELs only in A2. In 7% (5/68) of the clones, we determined that allele A1 had been used to repair A2 because i) there was no sign of the PAM silent mutation in either allele, ii) the c.2276G>T mutation was present in both alleles, and iii) the c.2299delG mutation was absent in both alleles. Lastly, 1.5% (1/68) clones showed no CRISPR/Cas9-induced modifications in the DNA.

The cloning and sequencing of the 68 clones revealed the presence of an SNP (rs111033281; c.2256T>C) in A1, in cis with the c.2276G>T missense variant that the inventors aimed at correcting.

To investigate if the SNP was also present before CRISPR/Cas9 modifications, we amplified, sub-cloned and sequenced the target region of the parental USH2A-RP-iPSC line and detected c.2256T>C in cis with c.2276G>T in A1 (FIG. 3C).

The presence of the SNP in A1 explained the reason the inventors got false positives after NcoI digestion of the PCR product, as the SNP is at the position of the NcoI site in A1 and A2 had been Cas9-modified, interfering with the NcoI recognition site.

These results demonstrated that a single mismatch between the gRNA sequence and the targeted locus may lead to the absence of recognition of the locus and thus to the absence of any correction.

Due to the presence of the SNP in A1, the inventors re-designed and cloned gRNA 1 to incorporate the SNP in the sequence (gRNA 1S) (SEQ ID NO: 7) so that it would recognize the missense variant allele A1 (FIG. 3D).

The inventors nucleofected the eSpCas9 (1.1)-gRNA 1S plasmid, together with ssODN 1, into the USH2A-USH-iPSC line and single-cell-sorted the EGFP-positive cells. The surviving clones (36/288) were expanded and screened for HDR events. The sequencing results showed that, in contrast to gRNA 1, gRNA 1S was able to recognize and induce cutting of not only the targeted allele A1 containing the SNP sequence, but also A2 (FIG. 3E).

Examples of the Cas9-induced modifications with gRNA 1S after cloning and sequencing are found in FIG. 3F.

In 5% (2/36) and 14% (5/36) of clones, the inventors detected partial repair by exclusive introduction of the PAM silent mutation in A1 and A2, or in A1 alone, respectively (FIG. 4C). In 14% (5/36) clones A2 had been used to repair Cas9-induced cleavage of A1, as suggested by i) the absence of the SNP, ii) the absence of the PAM silent mutation, iii) correction of the c.2276G>T mutation in A1, and iv) presence of the c.2299delG mutation in both A1 and A2. In 28% (10/36) and 19% (7/36) of the clones, the inventors found INDELs in the targeted allele A1, or in the non-targeted allele A2, respectively. In 17% (6/36) of clones, no Cas9-induced modifications could be detected. Lastly, in 3% (1/36) of clones, the inventors detected correction of the missense c.2276G>T variant in A1) and the presence of the c.2299delG only in A2 (FIG. 3G). The inventors believe though that the gRNA 1S had also recognized both alleles in this clone, as the PAM silent mutation was also homozygous.

Taken together, the inventors successfully achieved correction of the hypomorphic c.2276G>T mutation in the iPSC of a patient presenting with arRP.

### Example 4: Characterization of CRISPR/Cas9 Corrected iPSC

The inventors then examined whether the CRISPR-corrected iPSC clones generated maintained the pluripotency characteristics of their parental iPSC lines (31), which was not affected by the gene targeting process.

For this purpose, the inventors selected the corrected clones USH2A-USH-iPSC-B3B1 (homozygous correction of c.2299delG) and USH2A-RP-iPSC-MS3F7 (hemizygous correction of c.2276G>T) for detailed analysis.

Both corrected cell lines displayed the typical morphology of iPSC clones, comprising of tightly-packed cells surrounded by a distinct border (FIG. 4A). To rule out any major chromosomal rearrangements that may have been induced by nucleofection or by single cell culture, the inventors assessed their genomic integrity by the iCS-digital TM Pluri test (Assou, Bouckenheimer, & Vos, 2018) (2). The results indicated that both corrected lines retained normal genomic stability (FIG. 4B)

For the iPSC pluripotency studies and for the in vitro differentiation staining, cells were fixed using 4% PFA and permeabilized 0.1% Triton X-100 (Sigma-Aldrich). Non-specific binding was blocked with 1% BSA and 10% donkey serum (Millipore). Primary antibodies to detect NANOG, OCT3/4 and SOX2 (for pluripotency) and SMA, AFP and Nestin (for characterization of the three germ layers) were used at a 1:200 dilution and incubated overnight at 4° C. Fluorescence-conjugated secondary antibodies (Jackson ImmunoResearch) were incubated for 1 h at RT. Nuclei were stained with 0.2 μg/ml bisBenzimide (Sigma-Aldrich). Cells were observed using a Zeiss ApoTome 2 Upright wide-field microscope.

Immunofluorescence analysis revealed that the CRISPR/Cas9 corrected clones retained expression of typical pluripotency markers such as OCT3/4, SOX2 and NANOG (FIG. 4C).

In addition, the ability of these cell lines to give rise to the three embryonic cell layers was determined by an embryoid body (EB) assay.

Both cell lines were able to differentiate into the three germ layers as assessed by immunostaining of HNF4α for endoderm, Smooth Muscle Actin (SMA) for mesoderm and Glial Fibrillary Acidic Protein (GFAP) for ectoderm (FIG. 4D).

For the differentiation of the iPSC into the three germ layers, iPSC were dissociated with Accutase (Stemcell Technologies) and seeded on ultra-low attachment dishes for 2 days in E8 containing Y27632 StemMACS. At day 3, the medium was changed to DMEM/F12 (Gibco) supplemented with 20% Knockout serum replacement (Gibco), 1% penicillin-streptomycin (Gibco), 1% GlutaMax, 55 mM β-mercaptoethanol and 1% NEAA. At day 7, the embryoid bodies were seeded onto Matrigel-coated wells and culture for a further 10 days before immunofluorescence staining.

Taken together, the inventors successfully generated USH2A gene corrected iPSC displaying typical iPSC characteristics.

### Example 5: Evaluating the mRNA Expression Levels of USH2A in CRISPR Corrected iPSC

To determine whether there was a difference regarding USH2A expression in the mutant iPSC cell lines and the gene-corrected cells, the inventors evaluated the expression of USH2A at the mRNA levels.

USH2A is known to have two isoforms, a short isoform with 21 exons and a long isoform with 72 exons (37). Although both isoforms are present in the retina, the long isoform is the predominant form in photoreceptors (18). For this reason, the inventors designed primers targeting exon 39, which would recognize the long isoform exclusively, as well as exon 13, which would recognize the long and short isoforms, and evaluated USH2A mRNA levels by qPCR., following the previously described protocol.

Firstly, the iPSC of the patient with USH2 carrying c.2299delG in the homozygous state show expression levels of the long isoform that were 6-fold higher than wild type USH2A levels (FIG. 5A). Furthermore, the homozygous correction of the c.2299delG mutation in the USH2A-USH-iPSC-B3B1 line restored the USH2A mRNA expression levels back to those of wild type. This same profile was observed following indiscriminate amplification of both the long and short isoforms (FIG. 5B), thus confirming these phenomena.

These results suggested that the c.2299delG mutation has a specific effect on the accumulation of USH2A mRNA levels. Secondly, the iPSC of the patient with arRP who is compound heterozygous for the c.2276G>T missense mutation and the c.2299delG mutation, showed USH2A mRNA levels of the long isoform that were comparable to those of wild-type cells (FIG. 5C).

Furthermore, correction of the c.2276G>T mutation, which left the c.2299delG mutation in a hemizygous state, results in a 3-fold increase in USH2A mRNA levels when compared to wild-type or non-corrected iPSC. This same profile was observed following indiscriminate amplification of both the long and short isoforms (FIG. 5D), confirming a specific effect of the c.2299delG mutation on USH2A mRNA levels.

### Example 6: Evaluating the Structure of Retinal Organoids Originating from Patient's iPSC after CRISPR/Cas9 Mediated Correction of the c.2299delG Mutation

The inventors used an iPSC cell line (USH2A-USH-iPSC) from a patient presenting USH2 syndrome due to the homozygous mutation c.2299delG, which was obtained in the same manner as in Example 2.

The USH2A-USH-iPSC cell line was nucleofected with the eSpCas9 (1.1)-gRNA 2 plasmid in combination with ssODN 2, as described in Example 2. Forty-eight hours post-nucleofection, iPSCs were GFP single-cell sorted by fluorescence-activated cell sorting (FACS) and re-seeded in 96 well-plates.

The surviving iPSC colonies were matured and differentiated into retinal organoids.

iPSC were differentiated to retinal organoids using the following protocol. iPSC were cultured in Essential 8 medium (Thermo Fisher Scientific). When cells were 60-80% confluent, the medium was changed to Essential 6 medium (E6) (Thermo Fisher Scientific) and this was considered as day D0. At D1, the E6 medium was refreshed. At D2, the E6 medium was changed to E6 supplemented with N-2 supplement (E6-N2) (Thermo Fisher Scientific). The E6-N2 medium was refreshed 3 times per week for 28 days. At D28, the immature retinal organoids were manually dissected with a scalpel and cultured in BVA medium (DMEM-F12, non-essential amino acids and 0.1% penicillin-streptomycin (Thermo Fisher Scientific)) supplemented with basic fibroblast growth factor (b-FGF) and B-27 supplement for 1 week. The medium was refreshed 3 times per week. At D35, the medium was changed to BVA+FCS+Glutamax (BVA medium with 10% fetal calf serum (FCS) and Glutamax) supplemented with B-27. This medium was refreshed 3 times per week. At D85 of culture, the retinal organoids were cultured in BVA+FCS+Glutamax medium supplemented with B-27 without Vitamin A (VitA). The medium was refreshed 3 times per week until processing.

The same growth and differentiation methods were used on a wild-type (WT) iPSC line, which does not present a c.2299delG mutation (generated as described in 46, and on a USH2A-USH-iPSC line which was not nucleofected according to the invention, i.e. that was not corrected for the c.2299delG mutation.

Three different types of retinal organoids were obtained after 200 days of culture: WT organoids (positive control), USH2A-USH-iPSC-derived organoids (or USH2A-USH-organoid) (non-corrected) and CRISPR/Cas9 corrected USH2A-USH-iPSC-derived organoids (or CRISPR/Cas9-corrected USH2A-USH-organoid). This allowed the inventors to study and characterize possible defects associated with the c.2299delG mutation and Usher syndrome type 2.

The three cell lines differentiated into retinal organoids. Nonetheless, there was a difference in the lamination of the organoid and in the size of the brush border surrounding the organoid, which is a well-known feature of mature and structured organoids. The WT and the CRISPR/Cas9-corrected USH2A-USH-organoid presented a well-defined lamination and a brush border of 100 μm in length compared to an ill-defined lamination and a 50-μm brush border in the non-corrected USH2A-USH-organoid (FIG. 6).

Taken together, these results show that CRISPR/Cas9 correction of the c.2299delG mutation in retinal organoids corrects the defects observed in the lamination and maturation of the photoreceptors observed in the USH2A-USH-organoid.

### Example 7: Evaluating the Structure of Retinal Organoids Originating from Patient's iPSC after CRISPR/Cas9 Mediated Correction of the c.2276G>T Mutation

Here, the inventors used an iPSC cell line (USH2A-RP-iPSC) from a patient presenting non-syndromic RP due to a compound heterozygous mutation (c.2276G>T and c.2299delG), which was obtained in the same manner as in Example 3.

The USH2A-RP-iPSC cell line was nucleofected with the eSpCas9 (1.1)-gRNA 1S plasmid in combination with ssODN 1 as described in Example 3. Forty-eight hours post-nucleofection, iPSCs were single-cell sorted by GFP fluorescence-activated cell sorting (FACS) and re-seeded in 96 well-plates.

The surviving iPSC colonies were matured and differentiated into retinal organoids.

iPSCs were differentiated into retinal organoids, as described above for example 6 with slight modifications. At D42, the BVA+FCS+Glutamax medium was supplemented with Taurine. At D65, the BVA+FCS+Glutamax (+Taurine) medium was supplemented with B-27 without VitA and retinoic acid (RA). At D85, the retinal organoids were cultured in BVA+FCS+Glutamax (−VitA, +Taurine, +RA) supplemented with N-2. At D120, the organoids were cultured in BVA+FCS+Glutamax (−Vit A, +Taurine, +N-2, −RA). The medium was refreshed 3 times per week until processing.

The same growth and differentiation methods were used on a wild-type (WT) iPSC line, which does not carry any mutation (generated as described in46), and on a USH2A-USH-iPSC line which was not nucleofected according to the invention, i.e. that was not corrected for the mutation.

Three different types of retinal organoids were obtained after 150 days of culture: WT organoids (positive control), USH2A-RP-iPSC-derived organoids (or USH2A-RP-organoid) (non-corrected), and CRISPR/Cas9-corrected USH2A-RP-iPSC-derived organoids (or CRISPR/Cas9-corrected USH2A-RP-organoid). This allowed the inventors to study and characterize possible defects associated with the c.2276G>T mutation and retinitis pigmentosa (RP).

The three cell lines differentiated into retinal organoids. Nonetheless, there was a difference in the lamination and in the presence or absence of the brush border surrounding the organoid, which is a well-known feature of mature and structured organoids. The WT and the CRISPR/Cas9 corrected USH2A-RP-organoid presented a well-defined laminated structure and a brush border surrounding the whole surface of the organoid. In contrast, the USH2A-RP-organoid, showed a less well-defined lamina and did not present a brush border surrounding the organoid (FIG. 7).

Taken together, these results indicate that there is a delay in the formation of the laminated structure and brush border characteristic of a mature retina in the case of a USH2A-RP-organoid that is reversed by CRISPR/Cas9 correction.

SEQUENCES LISTING
SEQ ID NO: 1 (gRNA 1):
AGAATTTGTTCACTGAGCCA
SEQ ID NO: 2 (gRNA 2):
AATTCTGCAATCCTCACTCT
SEQ ID NO: 3 (gRNA 3):
CCCTGCCAGTGTAACCTCCA
SEQ ID NO: 4 (gRNA 4):
CTGAGCCATGGAGGTTACAC
SEQ ID NO: 5 (ssODN 1):
GGCTTAGGTGTGATCATTGCAATTTTGGATTTAAATTTCTCCGAAGC
TTTAATGATGTTGGATGTGAGCCCTGCCAGTGTAACCTCCATGGCTC
AGTGAACAAATTCTGCAATCCTCACTCTGGGCA
SEQ ID NO: 6 (ssODN 2):
TACAATTGGTGACATCTAACCCATAAAAGTTTTCTCTGCAGGTGTCA
CACTGAAGTCCTTTGGCTTCTTTTTTGCACTCACACTGGCCAGAGTG
AGGATTGCAGAATTTGTTCACTGAGCCATGGAG
SEQ ID NO: 7 (gRNA 1S):
AGAATTTGTTCACTGAGCCG

### REFERENCES

(1) Adikusuma, F., Piltz, S., Corbett, M. A., Turvey, M., McColl, S. R., Helbig, K. J., . . . Thomas, P. Q. (2018). Brief Communications Arising Inter-homologue repair in fertilized human eggs? Nature, 560(7717), E8-E9. https://doi.org/10.1038/nature23305
(2) Assou, S., Bouckenheimer, J., & Vos, J. de. (2018). Concise Review: Assessing the Genome Integrity of Human Induced Pluripotent Stem Cells: What Quality Control Metrics? Stem Cells, 36, 814-821. https://doi.org/10.1002/stem.2797
(3) Barnes, D. E. (2001). Non-homologous end joining as a mechanism of DNA repair. Current Biology, 11, R455-R457.
(4) Bassuk, A. G., Zheng, A., Li, Y., Tsang, S. H., & Mahajan, V. B. (2016). Precision Medicine: Genetic Repair of Retinitis Pigmentosa in Patient-Derived Stem Cells. Scientific Reports, 10(1038), 1-6. https://doi.org/10.1038/srep19969
(5) Baux, D., Blanchet, C., Hamel, C., Meunier, I., Larrieu, L., Castorina, P., . . . Claustres, M. (2014). Enrichment of LOVD-USHbases with 152 USH2A Genotypes Defines an Extensive Mutational Spectrum and Highlights. Human Mutation, 35(10), 1179-1186. https://doi.org/10.1002/humu.22608
(6) Berger, W., Kloeckener-Gruissem, B., & Neidhardt, J. (2010). The molecular basis of human retinal and vitreoretinal diseases. Progress in Retinal and Eye Research, 29(5), 335-375. https://doi.org/10.1016/j.preteyeres 0.2010.03.004
(7) Burnight, E. R., Gupta, M., Wiley, L. A., Anfinson, K. R., Tran, A., Triboulet, R., . . . Tucker, B. A. (2017). Using CRISPR-Cas9 to Generate Gene-Corrected Autologous iPSCs for the Treatment of Inherited Retinal Degeneration. Molecular Therapy, 25(9), 1-15. https://doi.org/10.1016/j.ymthe.2017.05.015
(8) Cong, L., Ran, F. A., Cox, D., Lin, S., Barretto, R., Hsu, P. D., Marraffini, L. A. (2013). Multiplex Genome Engineering Using CRISPR/VCas Systems. Science, 339(6121), 819-823. https://doi.org/10.1126/science.1231143.
(9) D'haene, B., Vandesompele, J., & Hellemans, J. (2010). Accurate and objective copy number profiling using real-time quantitative PCR. Methods, 50(4), 262-270. https://doi.org/10.1016/j.ymeth.2009.12.007
(10) Dimopoulos, I. S., Chan, S., MacLaren, R. E., & MacDonald, I. M. (2015). Pathogenic mechanisms and the prospect of gene therapy for choroideremia. Expert Opin Orphan Drugs, 3(7), 787-798. https://doi.org/10.1517/21678707.2015.1046434.
(11) Fuster-García, C., García-García, G., González-Romero, E., Jaijo, T., Sequedo, M. D., Ayuso, C., Aller, E. (2017). USH2A Gene Editing Using the CRISPR System. Molecular Therapy—Nucleic Acids, 8(September), 529-541. https://doi.org/10.1016/j.omtn.2017.08.003
(12) Garcia-Garcia, G., Aparisi, M. J., Jaijo, T., Rodrigo, R., Leon, A. M., Avila-Fernandez, A., Aller, E. (2011). Mutational screening of the USH2A gene in Spanish USH patients reveals 23 novel pathogenic mutations. Orphanet Journal of Rare Diseases, 6(October 2011), 65. https://doi.org/10.1186/1750-1172-6-65
(13) Ghazi, N. G., Abboud, E. B., Nowilaty, S. R., & Alkuraya, H. (2016). Treatment of retinitis pigmentosa due to MERTK mutations by ocular subretinal injection of adeno—associated virus gene vector: results of a phase I trial. Human Genetics, 135, 327-343. https://doi.org/10.1007/s00439-016-1637-y
(14) Jinek, M., Chylinski, K., Fonfara, I., Hauer, M., Doudna, J. A., & Charpentier, E. (2012). A Programmable Dual-RNA—Guided DNA Endonuclease in Adaptive Bacterial Immunity. Science, 337(6096), 816-821. https://doi.org/10.1126/science.1225829
(15) Kato-inui, T., Takahashi, G., Hsu, S., & Miyaoka, Y. (2018). Clustered regularly interspaced short palindromic repeats (CRISPR)/CRISPR-associated protein 9 with improved proof-reading enhances homology-directed repair. Nucleic Acids Research, 46(9), 4677-4688. https://doi.org/10.1093/nar/gky264
(16) Kimberling, W. J., Hildebrand, M. S., Shearer, A. E., Maren, L., Halder, J. A., Cohn, E. S., Smith, R. J. H. (2010). Frequency of Usher Syndrome in Two Pediatric Populations: Implications for genetic screening of Deaf and Hard of Hearing Children. Genetic in Medicine, 12(8), 512-516. https://doi.org/10.1097/GIM.0b013e3181e5afb8.
(17) Lin, S., Staahl, B. T., Alla, R. K., & Doudna, J. A. (2014). Enhanced homology-directed human genome engineering by controlled timing of CRISPR/Cas9 delivery. ELife, 3, e04766. https://doi.org/10.7554/eLife.04766
(18) Liu, X., Bulgakov, O. V, Darrow, K. N., Pawlyk, B., Adamian, M., Liberman, M. C., & Li, T. (2007). Usherin is required for maintenance of retinal photoreceptors and normal development of cochlear hair cells. Proceedings of the National Academy of Sciences of the United States of America, 104(11), 4413-4418. https://doi.org/10.1073/pnas.0610950104
(19) Maclaren, R. E., Groppe, M., Barnard, A. R., Cottriall, C. L., Tolmachova, T., Seymour, L., Seabra, M. C. (2014). Retinal gene therapy in patients with choroideremia: initial findings from a phase 1/2 clinical trial. Lancet, 6736(13), 1-9. https://doi.org/10.1016/50140-6736(13)62117-0
(20) Mali, P., Yang, L., Esvelt, K. M., Aach, J., Guell, M., Dicarlo, J. E., . . . Church, G. M. (2013). RNA-Guided Human Genome Engineering via Cas9. Science, 339(6121), 823-826. https://doi.org/10.1126/science.1232033
(21) Mandai, M., Fujii, M., Hashiguchi, T., Sunagawa, G. A., Ito, S., Sun, J., . . . Takahashi, M. (2017). iPSC-Derived Retina Transplants Improve Vision in rd1 End-Stage Retinal-Degeneration Mice. Stem Cell Reports, 8(1), 69-83. https://doi.org/10.1016/j.stemcr.2016.12.008
(22) Mcgee, T. L., Seyedahmadi, B. J., Sweeney, M. O., Dryja, T. P., & Berson, E. L. (2010). Novel mutations in the long isoform of the USH2A gene in patients with Usher syndrome type II or non-syndromic retinitis pigmentosa. Journal of Medical Genetics, 47, 499-506. https://doi.org/10.1136/jmg.2009.075143
(23) Millan, J. M., Aller, E., Jaijo, T., Blanco-kelly, F., & Ayuso, C. (2011). An Update on the Genetics of Usher Syndrome. Journal of Ophthalmology, 2011. https://doi.org/10.1155/2011/417217
(24) Paquet, D., Kwart, D., Chen, A., Sproul, A., Jacob, S., Teo, S., . . . Tessier-Lavigne, M. (2016). Efficient introduction of specific homozygous and heterozygous mutations using CRISPR/Cas9. Nature, 533(7601), 1-18. https://doi.org/10.1038/nature17664
(25) Pardo, B., Gomez-Gonzalez, B., & Aguilera, A. (2009). DNA double-strand break repair: How to fix a broken relationship. Cellular and Molecular Life Sciences, 66(6), 1039-1056. https://doi.org/10.1007/s00018-009-8740-3
(26) Pennings, R. J. E., Brinke, H., Weston, M. D., Claassen, A., Orten, D. J., Weekamp, H., Kremer, H. (2004). USH2A Mutation Analysis in 70 Dutch Families With Usher Syndrome Type II. Human Mutation, 730(October 2003), 1-8. https://doi.org/10.1002/humu.9259
(27) Ran, F. A., Hsu, P. D., Lin, C. Y., Gootenberg, J. S., Konermann, S., Trevino, A. E., . . . Zhang, F. (2013). Double nicking by RNA-guided CRISPR cas9 for enhanced genome editing specificity. Cell, 154(6), 1380-1389. https://doi.org/10.1016/j.cell.2013.08.021
(28) Renaud, J. B., Boix, C., Charpentier, M., De Cian, A., Cochennec, J., Duvernois-Berthet, E., Giovannangeli, C. (2016). Improved Genome Editing Efficiency and Flexibility Using Modified Oligonucleotides with TALEN and CRISPR-Cas9 Nucleases. Cell Reports, 14(9), 2263-2272. https://doi.org/10.1016/j.celrep.2016.02.018
(29) Richardson, C. D., Ray, G. J., DeWitt, M. A., Curie, G. L., & Corn, J. E. (2016). Enhancing homology-directed genome editing by catalytically active and inactive CRISPR-Cas9 using asymmetric donor DNA. Nature Biotechnology, 34(3), 339-344. https://doi.org/10.1038/nbt.3481
(30) Russell, S., Bennett, J., Wellman, J. A., Chung, D. C., Yu, Z. F., Tillman, A., . . . Maguire, A. M. (2017). Efficacy and safety of voretigene neparvovec (AAV2-hRPE65v2) in patients with RPE65-mediated inherited retinal dystrophy: a randomised, controlled, open-label, phase 3 trial. The Lancet, 390(10097), 849-860. https://doi.org/10.1016/S0140-6736(17)31868-8
(31) Sanjurjo-Soriano, C., Erkilic, N., Manes, G., Dubois, G., Hamel, C. P., Meunier, I., & Kalatzis, V. (2018a). Generation of a human iPSC line, INMi002-A, carrying the most prevalent USH2A variant associated with Usher syndrome type 2. Stem Cell Research, 33(November), 247-250. https://doi.org/10.1016/j.scr.2018.11.007
(32) Sanjurjo-Soriano, C., Erkilic, N., Manes, G., Dubois, G., Hamel, C. P., Meunier, I., & Kalatzis, V. (2018b). Generation of an iPSC line, INMi001-A, carrying the two most common USH2A mutations from a compound heterozygote with non-syndromic retinitis pigmentosa. Stem Cell Research, 33(November), 228-232. https://doi.org/10.1016/j.scr.2018.11.004
(33) Sanjurjo-Soriano, C., & Kalatzis, V. (2018). Guiding Lights in Genome Editing for Inherited Retinal Disorders: Implications for Gene and Cell Therapy. Neural Plasticity, 2018, 5056279. https://doi.org/10.1155/2018/5056279
(34) Slaymaker, I. M., Gao, L., Zetsche, B., Scott, D. A., Yan, W. X., & Zhang, F. (2016). Rationally engineered Cas9 nucleases with improved specificity. Science, 351(6268), 84-88. https://doi.org/10.1126/science.aad5227
(35) Sohocki, M. M., Daiger, S. P., Bowne, S. J., Rodriquez, J. A., Northrup, H., Heckenlively, J. R., . . . Sullivan, L. S. (2001). Prevalence of mutations causing retinitis pigmentosa and other inherited retinopathies. Human Mutation, 17(1), 42-51. https://doi.org/10.1002/1098-1004(2001)17:1<42::AID-HUMU5>3.0.CO;2-K
(36) Strouse, B., Bialk, P., Niamat, R. A., Rivera-torres, N., & Kmiec, E. B. (2014). Combinatorial gene editing in mammalian cells using ssODNs and TALENs. Scientific Reports, 4(3791). https://doi.org/10.1038/srep03791
(37) van Wijk, E., E Pennings, R. J., to Brinke, H., Claassen, A., Yntema, H. G., Hoefsloot, L. H., Kremer, H. (2004). Identification of 51 Novel Exons of the Usher Syndrome Type 2A (USH2A) Gene That Encode Multiple Conserved Functional Domains and That Are Mutated in Patients with Usher Syndrome Type II. Am. J. Hum. Genet, 74, 738-744. https://doi.org/10.1086/383096
(38) Yan, D., & Liu, X. Z. (2010). Genetics and pathological mechanisms of Usher syndrome. Journal of Human Genetics, 55(6), 327-335. https://doi.org/10.1038/jhg.2010.29
(39) Yu, W., Mookherjee, S., Chaitankar, V., Hiriyanna, S., Kim, J.-W., Brooks, M., Wu, Z. (2017). Nrl knockdown by AAV-delivered CRISPR/Cas9 prevents retinal degeneration in mice. Nature Communications, 8, 14716. https://doi.org/10.1038/ncomms14716
(40) Zallocchi, M., Binley, K., Lad, Y., Ellis, S., Widdowson, P., Iqball, S., Cosgrove, D. (2014). EIAV-based retinal gene therapy in the shakerl mouse model for usher syndrome type 1B: Development of UshStat. PLoS ONE, 9(4), e94272. https://doi.org/10.1371/journal.pone.0094272
(41) Zhang, Y., Long, C., Li, H., McAnally, J. R., Baskin, K. K., Shelton, J. M., Olson, E. N. (2017). CRISPR-Cpf1 correction of muscular dystrophy mutations in human cardiomyocytes and mice. Science Advances, 3(4), 1-10. https://doi.org/10.1126/sciadv.1602814
(42) Stewart M P, Shrei A., Ding X., Sahay G., Langer R. and Jensen K F (2016). In vitro and ex vivo strategies for intracellular delivery. Nature, 538(7624), 183-192. https://doi.org/10.1038/nature19764
(43) Slaymaker I M, Gao L, Zetsche B, Scott D A, Yan W X and Zhang F (2016). Rationally engineered Cas9 nucleases with improved specificity. Science, 351(6268) 84-8. https://doi.org/10.1126/science.aad5227
(44) Mali P, Esvelt K M, Church G M (2013). Cas9 as a versatile tool for engineering biology. Nature Methods, 10(10):957-63. https://doi.org/10.1038/nmeth.2649
(45) Wright A V, Nunez J K, Doudna J A (2016). Biology and Applications of CRISPR Systems: Harnessing Nature's Toolbox for Genome Engineering. Cell, 164(1-2):29-44. https://doi.org/10.1016/j.cell.2015.12.035
(46) Torriano S, Ekilic N, Faugère V, Damodar K, Hamel C P, Roux A F, Kalatzis V (2017). Pathogenicity of a novel missense variant associated with choroideremia and its impact on gene replacement therapy. Human Molecular Genetics, volume 26, issue 18, 3573-3584. https://doi.org/10.1093/hmg/ddx244
(47) Wang Y, Gao Y, He C, Ye Z, Gerecht S, Cheng L (2016). Scalable Production of Human Erythrocytes from Induced Pluripotent Stem Cells. https://doi.org/10.1101/050021
(48) Lapillonne H, Kobari L, Mazurier C, Tropel P, Giarratana M C, Zanella-Cleon I, Kiger L, Wattenhofer-Donzé M, Puccio H, Hebert N, Francina A, Andreu G, Viville S, Douay L (2010). Red blood cell generation from human induced pluripotent stem cells: perspectives for transfusion medicine. Haematologica, 95(10):1651-9. https://doi.org/10.3324/haematol.2010.023556.
(49) Dias J, Gumenyuk M, Kang H, Vodyanik M, Yu J, Thomson J A, Slukvin II (2011). Generation of red blood cells from human induced pluripotent stem cells. Stem Cells Dev, 20(9):1639-47. https://doi.org/10.1089/scd.2011.0078.
(50) Nayerossadat N, Maedeh T, Ali PA (2012). Viral and nonviral delivery systems for gene delivery. Advanced Biomedical Research, 1:27. https://doi.org/10.4103/2277-9175.98152.
(51) Pang Y, Tang L, Zhou Y (2017). Subretinal Injection: A Review on the Novel Route of Therapeutic Delivery for Vitreoretinal Diseases. Ophtalmic Research, 58(4):217-226. https://doi.org/10.1159/000479157.

1. An in vitro or ex vivo method for correcting at least one of the USH2A mutations selected among c.2276G>T and c.2299delG mutations, both in exon 13, in the genome of an individual's induced pluripotent stem cell (iPSC), comprising the steps of:
(i) providing to the cell a site-directed genetic engineering system by:
(a) providing to the cell at least one guide nucleic acid (gRNA) comprising at least one nucleic acid sequence selected from the group consisting of SEQ ID NO: 1, SEQ ID NO:2 and SEQ ID NO: 7;
(b) providing to the cell at least one Clustered regularly interspaced short palindromic repeats (CRISPR) associated nuclease; and
(c) further providing to the cell at least one donor nucleic acid that serves as a repair template for the mutated USH2A gene;
(ii) culturing the cell obtained at step (i) such that the at least one donor nucleic acid is integrated in the cell genome so as to correct the at least one USH2A gene mutations.

2. The method of claim 1, wherein the iPSC is derived from an in vitro processing of a cell previously collected from an individual having a genome bearing one or both of the USH2A gene mutations.

3. The method according to claim 2, wherein the individual having a genome bearing one or both of the USH2A gene mutations is an individual suffering from an inherited retinal dystrophy.

4. The method according to claim 1, wherein the at least one gRNA consists of at least one nucleic acid sequence selected from the group consisting of SEQ ID NO: 1, SEQ ID NO:2 and SEQ ID NO: 7.

5. The method according to claim 1, wherein the at least one donor nucleic acid that serves as a repair template is complementary to the strand not targeted by the gRNA.

6. The method according to claim 1, wherein the at least one donor nucleic acid that serves as a repair template is asymmetrical.

7. The method according to claim 1, wherein the at least one donor nucleic acid that serves as a repair template comprises at one end or at both ends, at least one modified terminal base.

8. The method according to claim 1, wherein the at least one donor nucleic acid that serves as a repair template is a ssODN that is complementary to the strand non-targeted by the gRNA, that is asymmetrical, and that comprises two phosphorothioate-modified terminal bases at both ends.

9. The method according to claim 1, wherein the at least one donor nucleic acid that serves as a repair template comprises at least one nucleic acid sequence selected from the group consisting of SEQ ID NO: 5 and SEQ ID NO: 6 or consists of at least one nucleic acid sequence selected from the group consisting of SEQ ID NO: 5 and SEQ ID NO: 6.

10. A genetically modified induced pluripotent stem cell (iPSC) obtainable by a method according to claim 1.

11. A pharmaceutical composition comprising at least one cell differentiated from a genetically modified iPSC according to claim 10, in a pharmaceutically acceptable medium.

12. (canceled)

13. A method for treating an inherited retinal dystrophy in an individual in need thereof comprising a step of administering to said individual a genetically modified induced pluripotent stem cell (iPSC) according to claim 10 or a pharmaceutical composition comprising at least one cell differentiated from the genetically modified iPSC.

14. A site-directed genetic engineering system for correcting at least one of the USH2A gene mutations selected from c.2276G>T and c.2299delG mutations, in the genome of a cell, of an individual in need thereof, comprising:
(i) at least one guide nucleic acid comprising at least one nucleic acid sequence selected from the group consisting of SEQ ID NO: 1, SEQ ID NO:2 and SEQ ID NO: 7;
(ii) at least one Clustered regularly interspaced short palindromic repeat (CRISPR) associated nuclease;
(iii) at least one donor nucleic acid that serves as a repair template for the USH2A gene; and
(iv) optionally at least one delivery vehicle comprising at least the elements of (i), (ii) and (iii).

15. The system of claim 14, wherein an individual in need thereof is an individual suffering from at least one inherited retinal dystrophy.

16. The system according to claim 14, wherein the at least one guide nucleic acid consists of at least one nucleic acid sequence selected from the group consisting of SEQ ID NO: 1, SEQ ID NO:2 and SEQ ID NO: 7.

17. The system according to claim 14, wherein the at least one donor nucleic acid that serves as a repair template is a ssODN that is complementary to the strand non-targeted by the gRNA, that is asymmetrical, and that comprises at one end or at both ends, at least one phosphorothioate-modified terminal bases.

18. The system according to claim 14, wherein the at least one donor nucleic acid that serves as a repair template comprises at least one nucleic acid sequence selected from the group consisting of SEQ ID NO: 5 and SEQ ID NO: 6, or consists of at least one nucleic acid sequence selected from the group consisting of SEQ ID NO: 5 and SEQ ID NO: 6.

19. The system according to claim 14, wherein the at least one delivery vehicle is selected from the group consisting of viral vectors and non-viral vectors.

20. The system according to claim 19, wherein the viral vectors are selected from the group consisting of retroviral vectors, adenoviral vectors, adeno-associated virus vectors, herpes simplex virus vectors, lentivectors, poxvirus vectors and Epstein-Barr virus vectors.

21. A method for treating an inherited retinal dystrophy in an individual in need thereof comprising a step of administering to said individual a system according to claim 14.

22. The method according to claim 1, wherein the Clustered regularly interspaced short palindromic repeats (CRISPR) associated nuclease is a CRISPR associated protein 9 (Cas9).

23. The method according to claim 22, wherein the CRISPR associated protein 9 (Cas9) is a high efficiency CRISPR associated protein 9 (eSpCas9 (1.1)).

24. The method according to claim 1, wherein the donor nucleic acid that serves as a repair template for the mutated USH2A gene is in the form of a single-stranded oligodeoxynucleic acid (ssODN).

25. The method according to claim 3, wherein the inherited retinal dystrophy is retinitis pigmentosa.

26. The method according to claim 25, wherein the retinitis pigmentosa is an isolated retinitis pigmentosa or a retinitis pigmentosa in association with hearing loss as part of Usher syndrome type 2.

27. The method according to claim 7, wherein the at least one modified terminal base is a phosphorothioate-modified terminal base.

28. The genetically modified induced pluripotent stem cell (iPSC) according to claim 10, wherein the c.2276G>T mutation has been corrected.

29. The method according to claim 13, wherein the inherited retinal dystrophy is a retinitis pigmentosa.

30. The method according to claim 29, wherein the retinitis pigmentosa is an isolated retinitis pigmentosa or a retinitis pigmentosa in association with hearing loss as part of Usher syndrome type 2.

31. The site-directed genetic engineering system according to claim 14, wherein the cell is a photoreceptor cell.

32. The system according to claim 14, wherein the Clustered regularly interspaced short palindromic repeats (CRISPR) associated nuclease is a CRISPR associated protein 9 (Cas9).

33. The system according to claim 32, wherein the CRISPR associated protein 9 (Cas9) is a high efficiency CRISPR associated protein 9 (eSpCas9 (1.1)).

34. The system according to claim 14, wherein the donor nucleic acid that serves as a repair template for the USH2A gene is in the form of a single-stranded oligodeoxynucleic acid (ssODN).

35. The system according to claim 15, wherein the inherited retinal dystrophy is a retinitis pigmentosa.

36. The system according to claim 35, wherein the retinitis pigmentosa is an isolated retinitis pigmentosa or a retinitis pigmentosa in association with hearing loss as part of Usher syndrome type 2.

37. The system according to claim 17, wherein the at least one donor nucleic acid that serves as a repair template comprises at one end or at both ends at least two phosphorothioate-modified terminal bases.

38. The system according to claim 20, wherein the viral vectors are selected from adeno-associated virus vectors.

39. The method according to claim 21, wherein the inherited retinal dystrophy is a retinitis pigmentosa.

40. The method according to claim 39, wherein the retinitis pigmentosa is an isolated retinitis pigmentosa or a retinitis pigmentosa associated with hearing loss as part of Usher syndrome type 2.
